(19) United States
(12) Patent Application Publication
(10) Pub. No.: US 2023/0132519 A1
(43) Pub. Date: May 4, 2023

(54) CIRCULAR METHODS FOR MANUFACTURING PRODUCTS FROM ALGAL BIOMASS AND ATMOSPHERIC CARBON REMOVAL WITH LONG-LIVED STORAGE USING ALGAE RESIDUAL BIOMASS USING PACKING AND SPREADED SINKAGE

(71) Applicant: Sinkco Labs, Paris (FR)

(72) Inventors: Victor Adrian Daniel Choquet, Paris (FR); Viji Donato Thomas, Paris (FR)

(21) Appl. No.: 17/975,985

(22) Filed: Oct. 28, 2022

Related U.S. Application Data
(60) Provisional application No. 63/273,818, filed on Oct. 29, 2021.

(51) Int. Cl. C12M 1/00; C12N 1/12; C12P 7/649

(52) U.S. Cl. CPC C12M 21/02; C12N 1/12; C12P 7/649

(57) ABSTRACT

The invention encompasses systems and processes for the extraction of, for example, consumer, animal, and industrial end products from algal biomass. In various embodiments, the invention encompasses ecofriendly methods for selective extraction and fractionation of algal products. These components include, but are not limited to, lipids, proteins, antioxidants, synthesized by algae, or the use of these compounds for further biochemical processes for synthesis of product compounds such as ethanol or biopolymer. The invention further encompasses method for the recovery algae residual biomass (ARB), its packing and transfer the deep-sea for carbon storage. These methods include energy efficient methods of filtration and packing and spread to the ocean surface to further allow reaching the seabed.

### CROSS-REFERENCE TO RELATED APPLICATION

This invention claims the benefit of and priority to U.S. Provisional Application No. 63/273,818, filed Oct. 29, 2021, and is incorporated herein by reference in its entirety.

### FIELD OF THE INVENTION

The invention encompasses systems and processes for the extraction of, for example, consumer, animal, and industrial end products from algal biomass. In various embodiments, the invention encompasses ecofriendly methods for selective extraction and fractionation of algal products. These components include, but are not limited to, lipids, proteins, antioxidants, and alcohols, synthesized by algae, or the use of these components for further biochemical processes for synthesis of end products such as ethanol or biopolymers. The invention further encompasses systems and methods for the recovery algae residual biomass (ARB), its packing and transfer to the deep-sea for carbon storage. These methods include energy efficient methods of filtration and packing and spread to the ocean surface to further allow reaching the seabed

### BACKGROUND OF THE INVENTION

Sustainable and environmentally conscious approaches are becoming increasingly popular within many industries such as food and transportation. As consumer's purchasing power and governments begin to push legislation in a more sustainable direction, this necessitates the introduction of green alternatives to the market. Algae has the potential to provide a sustainable solution to a variety of products across multiple industries that cater to the environmental demands of customers. Additionally, algae and the development of the blue economy is thought to offer solutions to rising anthropogenic issues in the years to come.

When compared to terrestrial farming, the production of algae and especially macroalgae possess many advantages over terrestrial crops. There are many advantages of algae farming, both economically and environmentally. Algae has a much higher biomass production rate per unit area when compared to terrestrial plants and does not require fresh water. Secondly, they are easier to depolymerize as they contain no lignin in their cell wall and have a low harvesting cost, making them an efficient choice for extracting components from the biomass. Furthermore, algae can be produced in their natural habitat, therefore contributing to the local ecology. Often, they do not require fertilizers or pesticides, thus reducing strain on resources and minimizing pollution. Finally, seaweed aquaculture beds (SABs) capture large amounts of CO₂ and have recently been proven to act as carbon sinks, making algal production a feasible tool for carbon sequestration.

Algae chemical groups include carbohydrates, proteins, lipids, and mineral ash. The majority of algae compounds are bioactive (especially antioxidants and pigments), possessing properties such as angiotensin-converting enzyme (ACE), anti-viral, anti-tumor, anti-coagulant, antilipidemic, hepatoprotective, immuno-stimulating, antidepressant and anti-anxiolytic activities. Today, algae chemical components are either (i) extracted and used directly as such, (ii) extracted and transformed to a new component (indirectly), or (iii) extracted and combined with non-algae-based compounds to formulate and manufacture consumer products. Herein we use the term algae derived products to englobe all compounds and products that imply or can imply algae. Today, algae are used in many industries such as food, cosmetic, pharmaceutical, and energetic industries. The main application sectors of algae today are the food, bioenergy, pharmaceutical, cosmetic, animal food and agriculture industries

The potential of algae to contribute to solutions for today's environmental problems has been well explored within the scientific community. Algae is now being integrated into water treatment processes, used as a food source, in cosmetic products, dietary supplements, agricultural and aquacultural industry. Many applications such as pharmaceuticals require more research to evaluate the presence of toxic compounds combined with beneficial ones. The exploration of algae as a source of bioethanol has shown promise in previous trials but is still difficult to compete with fossil energy production costs. Algae has been traditionally farmed in Asia for centuries but now other areas of the world including Europe and the U.S. are increasing the number of algae farms and cultivation for use in various industries.

A carbon footprint (CF) is usually related to the total amount of greenhouse gases (including carbon dioxide and methane) released into the atmosphere, because of the specific activities of an individual, community or organization. For this, several scopes can be used for a product and define the extent of emissions considered for establishing the overall CF on the product (usually cradle to gate or cradle to grave model). The ISO 140 (especially ISO 140 40 and ISO 140 44 standard) is the international standard for product CF. The average American's carbon footprint is 16 tones, while the global average is 4 tones.

A carbon offset relates to the amount of carbon emissions avoided, reduced, or stored during a process, which are either used by certain companies to directly offset carbon and sell it under the form of credits (i) or use carbon offsets to balance the CF of their product manufacturing or company emission (ii). The Oxford Principal for carbon offset classifies carbon offset into five types according to the methods used for the offset and its impact. Type I and II offsets correspond to reduced emissions. Type III, IV and V use carbon removal. Whether the carbon is stored or not and if so, for what duration defines the impact of each type.

The definition of carbon negative is not yet defined to a universal standard; therefore, products today can claim carbon negativity based on their own definition. Clarification of this term (such as the Oxford carbon offset's classification) will be soon legally defined by governments to ensure product impact verification and restrict marketing misusing terminology. Carbon negative herein refers to the long-term removal of carbon (type V offset). This allows the differentiation between other products that aim to remove carbon from the atmosphere into carbon stores such as living biomass.

New strategies to sequester carbon via direct CO₂ capture are being developed worldwide. The ocean already contains elements that naturally sequester carbon such as marine plants in the form of seagrass, saltmarshes, and mangroves. These are known as blue carbon mitigators. Recent studies show that algae also play a role in global blue carbon sequestration. A recent study found that 24% of algae globally ends up at the bottom of the ocean, sequestering 557 million tons of CO₂ yearly, which is equivalent to the yearly emissions of 64 million U.S. households. Few projects have aimed to enhance the impact of this natural phenomenon to sequester carbon, but interest is beginning to increase in this area. Another study used pumps to sink Sargassum muticum to the bottom of the ocean in order to sequester this source of carbon from the Caribbean Sea. In 2019, a company called Running Tide started growing kelp on biodegradable buoys that detach and sink to the bottom of the ocean.

After the extraction of the initial product, the process results in algal residual biomass (ARB). This remaining biomass is mostly treated as waste. Scientific studies in the last ten years reveal the high interest in using the ARB for other purposes to maximize the algae compounds value in a cascade of compounds. This is referred to today as the blue circular economy. Extensive research is carried out on the dual extraction of compounds and biofuel to reduce the overall cost. Many studies successfully reported the use of the bioethanol production's byproducts as animal feed. A similar process is today applied in the distillery industry with distillers' grain and soluble alcohols. Overall, today very few algal cascade systems are applied to industries.

Marine plants are mainly composed of carbohydrates, lipids, proteins, uronic acids, and ashes. Most of these compounds contain atmospheric carbon. After extraction, the algal residual biomass still contains carbon, therefore it can be used for a single targeted compound product or as part of the cascade model being developed today. The current inventors first utilized a part of the algal biomass to produce consumer products and used the remaining part for permanent carbon removal, therefore making the product manufacturing process carbon negative. This provides everyday consumers the opportunity to enjoy their products whilst participating in a solution to climate change. An overview of the claimed process from algae harvesting to the sequestration of CO₂ in the ocean is represented in FIG. 3.

In the early mid-century there was a scientific effort conducted to answer the question of whether injecting liquid CO₂ in the ocean could reduce anthropogenic emissions. During this period of research, the question of the depth at which liquid CO₂ would not “leak” back to the surface was assessed. The findings commonly agreed on a depth of 1000-1500 meters to be sufficient. Today, using the ocean to sequester carbon is seeing a rebound since several studies have brought to light the recently quantified amount of carbon reaching the deep sea originating from marine plants growing in the surface and in SABs. Instead of injecting a non-native compound into the ocean, as it was first proposed with CO₂, introducing algae residue acts as enhancing a natural process. This is considered today as morally acceptable depending on the method used. When algae die, it is partially consumed by surface microorganisms, and ARB remaining sinks to the bottom of the ocean.

Sinking algae biomass to a depth of 1500 m or below is widely accepted by the scientific community to be considered stored on a close to permeant time scale. This agreement bases itself on the many studies carried out on liquid CO₂, which established that CO₂ leaking back to the surface was null at a depth of 1000-1500. Organic carbon originating from algae is denser than liquid CO₂, using the same 1000-1500 m depth is sufficient for this carbon to not leak back to the surface and hence be sequestered on a near permanent timescale. The particulate organic matter (“POC”) reaching the bottom of the ocean varies between 0.5 to 5 g C m⁻². Ya-1 with the value being below 1 g C. m⁻². A recent study predicted a 40-55% decline of that flux by 2100 due to climate change pressure, creating a potential starvation risk for deep-sea organisms.

It should be noted that purposefully sinking organic matter in the ocean has a prerequisite to be evaluated and is subject to acceptance of a permit from the authorities of the country concerned. This is an extensive process as several evaluations must be carried out, such as pilot experimentation and monitoring of its impact on deep sea ecosystem.

Most current technologies directed towards carbon sequestrations are created with the purpose to sell the sequestered carbon in the form of credits. The invention includes systems and methods developed a technology to manufacture marine based carbon negative compounds and products. The invention also includes selling the end product's net carbon value under the form of credits to decrease the green premium cost of certain respective product manufacturing processes.

Extracting several compounds of the algae reduces the carbon quantity of the ARB dedicated for the offset process and reduces the overall carbon negative potential of the process. This invention differentiates itself from the approach of circular economy that is characterize generally with the (maximization of) cascade extractions of several compounds from the initial algae biomass.

Kodukula et al. (U.S. Pat. No. 9,593,300) described an apparatus that allows the production of carbon negative ethanol through the use of a specific machine located under the water to achieve this aim. Furthermore, this sequestration is based on the organic matter growth. It depends on carbon removed from the atmosphere and transferred to the final product, with no carbon storage process, hence making the carbon offset method employed type I carbon sequestration.

Rampolla et al. (U.S. Pat. No. 2021/0144935) discloses the use of a personal carbon offset block using mostly land plant biomass (seaweed is also mentioned in embodiment) to be packed and bound together in a rigid form. The carbon in the biomass is therefore stored semi permanently and can be used for garden wall construction and similar applications. The block manufacturing described in Rampolla et al. is not a consumer product nor consumer good but used as a personal direct carbon capture and storage method with suggestions for use of this block in recycling. This carbon storage is not permanent and therefore relates more to type IV carbon offset: carbon removal with a short life duration. Rampolla et al. aims for a high rigidity to allow structural integrity while the present invention aims for a semi solid short term non dissolution form. Finally, Rampolla et al. does not mention placing these blocks in the ocean.

It is recommended by the U.S. federation IPCC and the environmental change institute (University of Oxford) to use appropriate terms when talking about carbon sequestration. Indeed, each term can lead to a misleading understanding. Many patents or inventions claim to sequester carbon but do not give a detailed explanation of the classification of the offset used in their technology and claims. Some even claim long term sequestration while the truth is otherwise.

In this invention, the type of offset implicated in the invention is classified so that it shows a clear distinction between other similarly aimed technology and so that it will enable this invention to fit the future legislation on the classification of offsets currently being written by the US government.

### SUMMARY OF THE INVENTION

The invention generally encompasses systems and methods for extracting products of varying polarities from an algal biomass material. In particular, embodiments described herein concern extracting various products including lipids, proteins, alcohols, etc. of varying polarities from an algal biomass using solvents of varying polarity and/or a series of filters. In some embodiments, the filter is a microfilter. In certain embodiments, the methods and systems provide processes to sustainably manufacture consumer goods in a more performant approach than old previous methods. In certain embodiments, the methods and systems remove and permanently store CO₂ from the atmosphere. In certain embodiments, the systems and methods emit less CO₂, require less fresh water, and do not require deforestation leading to loss of diversity.

Generally, the invention describes a system and manufacturing technique of processing algae products and algae derived products that:

a) reduce the net overall carbon footprint of the product.

In certain embodiments, the manufacturing process include two major consecutive parts where:

a) a portion of the algae biomass is used for the manufacturing of a product.

b) the remaining portion of the algae is used for removal and permanent storage of atmospheric carbon.

In various embodiments, algae include carbohydrates, lipids, proteins, and minerals which enable a broad scope of product manufacturing. In various embodiments, the manufacturing of a single or plurality algae products target one or several classes of compounds (a), while the other compounds (e.g., ARB) are used for (b).

The invention generally encompasses the overall goal of algae product manufacturing using one or several classes of algae chemical compounds while using the others for carbon storage to reduce or negate the carbon emissions of the product manufacturing process. The carbon storage process mimics and enhances the natural carbon mitigation feature of algae where algae residual biomass is returned to the deep sea, and the carbon it contains, initially captured from the atmosphere is stored on a close to permanent time scale.

In certain embodiments, the manufacturing steps include: (i) obtaining algal biomass (ii) the processing of algae into one or several products (iii) the recovery of the ARB, (iv) the transformation and shaping of the ARB, and (v) storage of ARB.

In certain embodiments, micro or macro algae are collected from natural habitats, cultivated, or purchased. Algae is either fresh or dried.

In certain embodiments, algae biomass is subject to ecofriendly chemical physical processes to extract one or several algae compounds. The algae extracted compound is used as a product.

In certain embodiments, the unused algae compounds (ARB) are recovered as a solid with moisture content ranging 1-95%.

In certain embodiments, the ARB is packed using packaging fiber to an undefined soft mass shape of volume ranging 500 mm³ to 15 m³. In certain embodiments, the packed ARB density when submerged in seawater is superior to the density of the seawater and therefore sinks. In certain embodiments, the pre dissolution time of the ARB pack is inferior to its sinking time to sea floor.

In certain embodiments, the ARB packs are transported from the manufacturing facility to an ocean zone with depth lower than 1500 m and deposited. The ARB packs sank on the ocean floor possess a carbon content between 2-40,000 g C/m² and correspond to the amount of carbon stored close to infinite time.

In various embodiments, the simultaneous use of algae biomass for a product manufacturing and carbon permanent storage results in a reduced carbon emission of the algae product or algae derived product.

In various embodiments, the systems and methods can include various combinations of four general components: (i) agricultural production; (ii) biofuel production; (iii) agricultural residue utilization; and (iv) greenhouse gas accounting and/or sustainability assessment, in which utilization of a fraction of the biomass (e.g., agricultural residue) provides emissions accounting credits (e.g., carbon credits) and/or sustainability benefits to be associated with the product. These components can be interrelated and/or integrated (e.g., in a single supply/production chain).

In other embodiments, the invention encompasses using a single solvent and water to extract and fractionate components present in an algal biomass material. In other embodiments, these components include, but are not limited to, proteins, polar lipids, and neutral lipids, alcohols, etc. In still other embodiments, more than one solvent is used. In still other embodiments, a mixture of solvents is used. In other embodiments, the invention includes a fermentation process including preferably a catalytic enzyme.

In some embodiments, the methods and systems described herein are useful for extracting coproducts of lipids from an algal biomass material. Examples of such coproducts include, without limitation, proteinaceous material and carotenoids. Embodiments of the present invention allow for the simultaneous extraction and fractionation of algal products from algal biomass in a manner that allows for the production of both fuels, cosmetic products, and nutritional products.

Under one embodiment of the invention, a method for extraction with fractionation of oil and proteinaceous material from algal biomass material is provided.

In another embodiment, the invention encompasses systems and method of selectively removing products from an algal biomass comprising substantially intact algal cells includes combining an algal biomass and at least one solvent, to generate an extraction mixture, the extraction mixture including a substantially solid phase and a liquid phase, separating at least a portion of the liquid phase of the extraction mixture from the substantially solid phase.

In other embodiments, the solvent comprises a water miscible or water immiscible solvent. In some embodiments, the solvent comprises two water miscible or two water immiscible solvents. In other embodiments, the solvent set comprises one or more water miscible solvents and one or more water immiscible solvents. In still other embodiments, a first, second and/or third extraction mixture is heated to a temperature below its boiling point. In further embodiments, the extraction mixture is under a pressure greater than atmospheric pressure. In some embodiments, the at least one solvent comprise(s) one or more amphipathic solvents. In still further embodiments, at least one water miscible solvents is selected from the group consisting of methanol, ethanol, isopropanol, acetone, ethyl acetate, and acetonitrile. In other embodiments, at least one of a first, second, and third solvents comprises ethanol. In still other embodiments, the solvent set is added to the biomass in a 1:1 weight/weight ratio.

In other embodiments, the cells comprising the algal biomass are not dried or disrupted. In yet another aspect, the algal biomass is unfrozen. In another embodiment of the invention, the method further comprises adjusting the pH of at least one of the extraction mixtures to optimize protein extraction. In still other embodiments of the invention, the algal biomass is simultaneously at least partially dewatered while products are selectively extracted from the algal biomass.

In certain embodiments, the invention encompasses systems and method of selectively separating products from a wet algal biomass comprising:

a. providing a wet algal biomass;

b. adding a catalytic amount of an acid, preferably an organic acid;

c. pretreating the wet algal biomass under hydrothermal conditions;

d. mixing the mixture of (c) in the presence of an enzyme to provide one or more fermentable sugars;

e. separating the solid algal residual biomass (ARB) and liquid phases;

f. distilling the liquid phase to produce hydrous and anhydrous ethanol products;

g. filtering the remaining liquid phase by ultrafiltration to recover additional solid ARB, in which the total ARB is air dried; and

h. packing and transporting the ARB to deep ocean disposal area;

wherein the method is carbon neutral or carbon negative.

In certain embodiments, the enzyme is used simultaneously with the Saccharomyces cerevisiae yeast

In certain embodiments, the ARB comprises unfermented sugars, uronic acids, proteins, and others insoluble residue.

In certain embodiments, the liquid phase comprises ethanol, higher ethers, lipids, minerals, and uronic acid.

In certain embodiments, the acid is acetic acid.

In certain embodiments, the hydrothermal conditions include adding water and heating to a temperature of about 80° C.

In certain embodiments, the amount of ethanol produced is about 0.1 g of ethanol per g of dry algal biomass.

In certain embodiments, the wet algal biomass was ground into particles before adding an acid.

In certain embodiments, the water and algal biomass were mixed at a consistency 10% w/v in a stainless-steel pressure bioreactor fermenter.

In certain embodiments, high temperature increases the catalytic action of hydronium ions and organic acid present to degrade the algal biomass.

In certain embodiments, the enzymatic blend was used to hydrolyze the polysaccharide to fermentable sugars.

In certain embodiments, the hydrothermal pretreatment was used to disrupt the cellular wall of the feed's cells for the recovery of glucan.

In certain embodiments, the heating of the algal biomass and water mixture is completed at a temperature of 120° C.

In certain embodiments, the separating of the solid algal residual biomass and liquid phases is done using a screw press.

In certain embodiments, the hydrous and/or anhydrous ethanol is isolated in a purity of greater than about 90%.

In certain embodiments, the hydrous and/or anhydrous ethanol is isolated in a purity of greater than about 95%.

In certain embodiments, the hydrous and/or anhydrous ethanol is isolated in a purity of greater than about 97%.

In certain embodiments, the mixture is held under a pressure greater than or equal to atmospheric pressure for a period of time.

In certain embodiments, the period of time is at least 60 minutes.

In certain embodiments, the algal biomass is unfrozen.

In certain embodiments, the methods further comprise adjusting the pH of the mixture to optimize alcohol extraction.

In certain embodiments, the methods further comprise repeating the sequence of combining and separating steps at least one more time.

In certain embodiments, the algal biomass is simultaneously at least partially dewatered while products are selectively extracted from the algal biomass.

In other embodiments, the invention encompasses a system and method for manufacturing algae compounds and related products and algae derived compound products that reduce, neutralize, or negate the overall carbon footprint of those products.

In certain embodiments, the systems and methods include farming a plurality of algae or wild harvesting a plurality of algae. In certain embodiments, the systems and methods include transforming the plurality of algae compounds by a plurality of chemical physical process into a plurality of compound products. In certain embodiments, the systems and methods include recovery of unused algae residual compounds obtained as a byproduct of the processing of the plurality of harvested algae. In certain embodiments, the systems and methods include packing of the (ARB) by binding gel, packeting fiber, or drying (with optional compression process) and transporting the ARB packs by sea ways, roadways, rails ways, air ways and its sinking to an ocean zone where the depth is below 1000 m. In certain embodiments, the systems and methods include tracing and recording in a database all the greenhouse gas emissions from each of the processing steps associated with the manufacturing process.

In certain embodiments, the systems and methods of algae product manufacturing apply to any industry sectors such as bioethanol for various industries, which comprises fuel, consumer products, spirits, perfume; cosmetics, which includes face cream, hair gel, any types of cream for skin, hair, shampoos; and other consumer products.

In certain embodiments, the systems and methods monitor the product carbon footprint status (i.e., neutral, negative and reduced) established by the current and future international ISO standards or the corresponding current or future country's legislation in which this manufacturing method is applied.

In certain embodiments, the systems and methods include energy saving methods for all steps, which result in increased negation power of the product carbon footprint according to the standards.

In certain embodiments, the systems and methods include algae including, but not limited to, microalgae, macroalgae, and blue green algae: cyanobacteria; freshwater algae species, in a separated or simultaneous cultivation system (multispecies cultivation).

In certain embodiments, the systems and methods include farming the algae in non-arable land or in the ocean to prevent the withdrawal of arable spaces for food crops.

In certain embodiments, the systems and methods include farming the algae with non-intensive techniques including, but not limited to, wild cultivation, ocean aquaculture, open ponds, collaboration with fish farm in circular aquaculture system (other aquaculture species growth: intended to optimize the overall carbon footprint of the product (according to its ISO inventory scope).

In certain embodiments, the systems and methods include farming the algae with intensive techniques intended to optimize productivity including ocean aquaculture, open ponds, vertical or horizontal tubular photobioreactor, flat panel airlift photobioreactor, and bubble column photobioreactor.

In certain embodiments, the systems and methods include farming the algae in sea water or freshwater.

In certain embodiments, the systems and methods include genetically modifying and/or use of genetically modified plurality of algae to improve the photosynthetic efficiency.

In certain embodiments, the systems and methods include strain selective and/or use of strain selective plurality of algae to improve the photosynthetic efficiency.

In certain embodiments, the systems and methods include processes in which any step can be located totally or separately located on land, coastal or offshore area, on the surface water or above on platforms.

In certain embodiments, the systems and methods include the isolation and purification of a plurality of compounds present in the plurality of harvested algae.

In certain embodiments, the systems and methods include the use of the plurality of algae compounds isolated, purified or extracted by chemical and/or physical process for further chemical-physical-chemical process into a plurality of algae-derived compound products.

In certain embodiments, the systems and methods include the use of the plurality of algae-derived compound products in order to make a plurality of commercial products.

In certain embodiments, the systems and methods include testing the safety of the formulated plurality of compounds which are intended for human, animals or plants consumption or use.

In certain embodiments, the systems and methods include the recovery of unused algae residual compounds in any steps of the process.

In certain embodiments, the systems and methods include a combination of precipitation, filtration, microfiltration, ultrafiltration, nanofiltration, reverse osmosis, centrifugation, screw pressing, hydraulic pressing, sedimentation, flocculation, coagulation, hydro cyclone separation, auger pressing, drying, air classification, coagulation, evaporation, sun drying, airdrying, dissolved air flotation and/or granular filtration.

In certain embodiments, the systems and methods include the drying of the ARB with energy-saving techniques intended to reduce the greenhouse gas emissions including, for example, spray drying, sun-drying, freeze-drying, tray drying, and/or drum rotary drying.

In certain embodiments, the systems and methods include the compression of the wet or dried ARB with energy-saving techniques intended to reduce the greenhouse gasses emissions including, for example, hydraulic press, forging press, crank press, eccentric press, knuckle joint press, extruder, pelletizer, grinder, and/or shredder.

In certain embodiments, the systems and methods include mixing of the wet or dried ARB with a plurality of algae-derived binding agents especially algae-derived bindings agents.

In certain embodiments, the systems and methods include mixing of the wet or dried ARB with a construction material including mortar, concrete, or cement.

In certain embodiments, the systems and methods include packing form compostable film, organic packing, and/or wooden crate.

In certain embodiments, the systems and methods include the transportation of the ARB with energy-saving methods intended to reduce the greenhouse gas emissions from transportation by boat and transportation by truck.

In certain embodiments, the systems and methods include the deposit of the ARB with an operation performed via sea vessel and undersea vessels.

In certain embodiments, the systems and methods include the deposit of the ARB in a geologically stable land area that can be artificially prepared or naturally available.

In certain embodiments, the systems and methods include the optimization of CO₂ and reduction of emissions by using sensors on equipment.

In certain embodiments, the method of manufacturing ethanol and ocean carbon dioxide removal (CDR) generates a carbon offset by the removal of atmospheric carbon dioxide and permanent disposal of the residual algal biomass, while simultaneously avoiding production of atmospheric carbon dioxide typically emitted during the manufacture of ethanol.

In certain embodiments, the method of manufacturing ethanol and ocean carbon dioxide removal generates a carbon offset by the removal of atmospheric carbon dioxide and permanent disposal of the residual algal biomass, while simultaneously avoiding production of atmospheric carbon dioxide typically emitted during the manufacture of ethanol.

### BRIEF DESCRIPTION OF THE FIGURES

FIG. 1 provides an illustrative representation of the general process to produce algae-derived products including the storage of algal residual biomass (ARB).

FIG. 2 provides an illustrative diagram showing the treatment and storage of the ARB obtained as a byproduct of the bioprocess

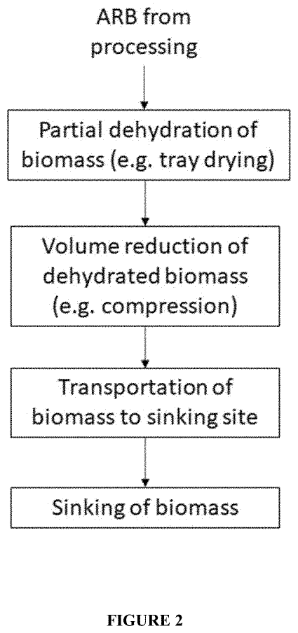

FIG. 3 provides an illustrative schematic flow chart representing the majors steps of the process: Atmospheric CO₂ (1) is absorbed by algae (2) and assimilated in its biomass carbon content. Algae (2) are collected and used as raw material to manufacture compounds and products in the manufacturing plant (3). The Algae residual biomass from the process is recovered, packed (4), and transported offshore on a vessel (5). The packed ARB (5) is sunk at or below 1500 meters depth (6).

FIG. 4 provides the algae residual biomass at 85% moisture homogenized with agar to from a solid gel as explained in embodiment b) step (iv) of the section 4.

FIG. 5 illustrates a diagram showing the process to produce carbon-negative bioethanol.

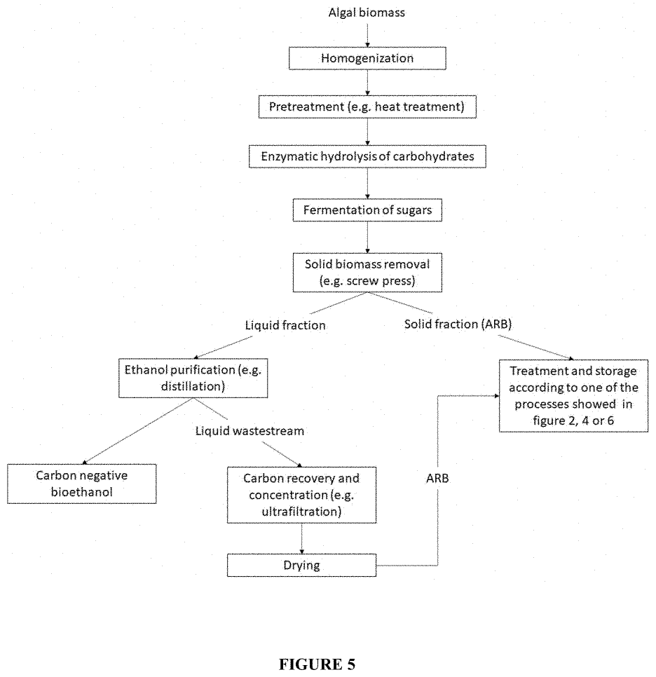

FIG. 6 illustrates diagram showing the process to produce carbon-negative protein powder.

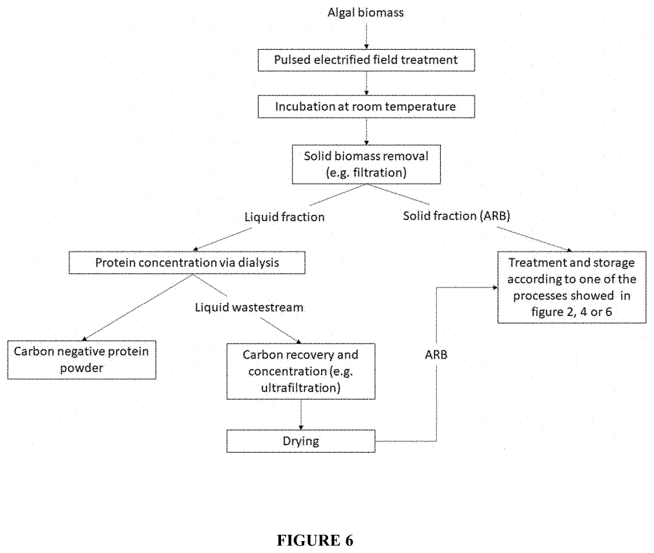

FIG. 7 illustrates a histogram of the maximum glucose value obtained at time t after enzymatic hydrolysis expressed as percentage of the initial pretreated U. lactuca biomass used.

FIG. 8 illustrates a gas chromatograph with SPME showing the impurities of the seaweed ethanol sample purified with 3 steps pervaporation from a pot still followed by column on 96% ABV seaweed ethanol sample.

FIG. 9 illustrates a histogram of TPC expressed with GAE equivalent obtained for each liquid phase of pretreated biomass by autohydrolysis with Severity S0=1.82 (Tmax=130° C.) to S0=3.16 (Tmax=170° C.).

FIG. 10 illustrates a histogram of Trolox Equivalent measure obtained in the liquid phase of each pretreatment severity from S0=1.82 (Tmax=130° C.) to S0=3.16 (Tmax=170° C.).

FIG. 11 illustrates the classification of coastal zones.

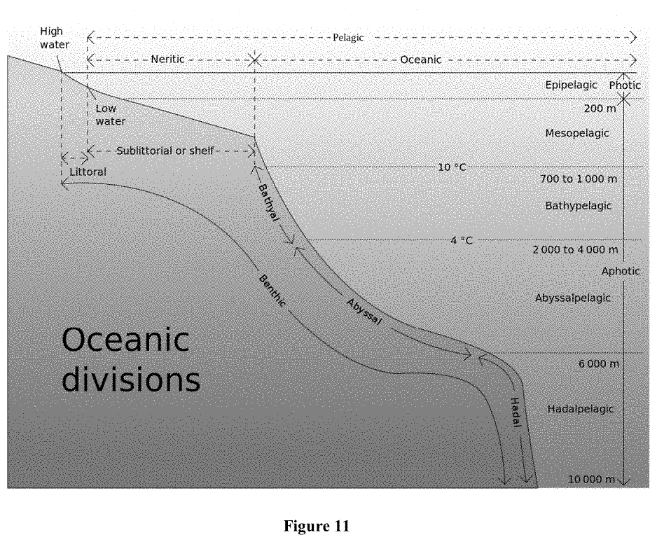

### DETAILED DESCRIPTION OF THE INVENTION

### Definitions

The term “about” or “approximately,” as used herein, are defined as being close to as understood by one of ordinary skill in the art, and in one non-limiting embodiment the terms are defined to be within 10%, preferably within 5%, more preferably within 1%, and most preferably within 0.5%.

The term “albumin proteins” as used herein refers to water soluble proteins.

The term “algal biomass” as used in this specification means any composition comprising algae. The algal biomass may be partially de-watered, i.e. some of the water has been removed during the process used to harvest the algae, for example during aggregation, centrifugation, micro-screening, filtration, drying or other unit operation. The algal biomass may also comprise dried algae. The raw material may also comprise additional biomass derived from other sources and may therefore implicitly comprise, without express statement of, “other contributing biomass” which may be biomass derived from other sources, such as for example biomass from cellulosic sources. Saltwater algal cells include, but are not limited to, marine and brackish algal species. Saltwater algal cells are found in nature in bodies of water such as, but not limited to, seas, oceans, and estuaries. Non-limiting examples of saltwater algal species include Nannochloropsis sp., Dunaliella sp. Freshwater algal cells are found in nature in bodies of water such as, but not limited to, lakes and ponds. Non-limiting examples of freshwater algal species include Scendescemus sp., Haemotococcus sp.

The term “algal cake” as used herein refers to a partially dewatered algal culture that lacks the fluid properties of an algal paste and tends to clump. Generally, an algal cake has a water content of about 60% or less.

The term “algal culture” as used herein refers to algal cells in culture medium.

The term “algal paste” as used herein refers to a partially dewatered algal culture having fluid properties that allow it to flow. Generally, an algal paste has a water content of about 90%.

The term “animal feed” as used herein refers to algae-derived substances that can be consumed and used to provide nutritional support for an animal.

The term “biodiesel” as used herein refers to methyl or ethyl esters of fatty acids derived from algae.

The term “biofuel” as used herein refers to fuel derived from biological source. Non-limiting examples include biodiesel, jet fuel, diesel, jet fuel blend stock and diesel blend stock.

The term “biomass” as used in this specification means any material of biological origin, including that having undergone processing, but not including that which has been fossilized.

The term “comprising” as used in this specification means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “detergents,” when used in connection with polar lipids, as used herein refers to glycolipids, phospholipids and derivatives thereof.

The term “dewatered” as used herein refers to the removal of at least some water.

The term “diffusate” or “permeate” as used herein may refer to material that has passed through a separation device, including, but not limited to a filter or membrane.

The term “effective,” as used herein, means adequate to accomplish a desired, expected, or intended result.

The term “enriched”, as used herein, shall mean about 50% or greater content.

The term “food additives”, when used in connection with polar lipids, as used herein refers to soy lecithin substitutes or phospholipids derived from algae.

The term “human food” as used herein refers to algae-derived substances that can be consumed to provide nutritional support for people. Algae-derived human food products can contain essential nutrients, such as carbohydrates, fats, proteins, vitamins, or minerals.

The term “impurities”, when used in connection with polar lipids, as used herein, refers to all components other than the products of interest that are coextracted or have the same properties as the product of interest.

The terms “inhibiting” or “reducing” or any variation of these terms, as used herein, includes any measurable decrease or complete inhibition to achieve a desired result.

The term “lubricants”, when used in connection with polar lipids, as used herein refers to hydrotreated algal lipids such as C16-C20 alkanes.

The term “neutral lipids” or any variation thereof, as used herein, includes, but is not limited to, triglycerides, diglycerides, monoglycerides, carotenoids, waxes, sterols.

The term “non-glycerin matter” as used herein refers to any impurity that separates with the glycerin fraction. A further clean up step will remove most of what is present in order to produce pharmaceutical grade glycerin.

The term “nutraceutical” as used herein refers to a food product that provides health and/or medical benefits. Non-limiting examples include carotenoids, carotenes, xanthophylls such as zeaxanthin, astaxanthin, and lutein.

The term “oil” as used herein includes compositions containing neutral lipids and polar lipids. The terms “algae oil” and “algal oil” as used herein are used interchangeably.

The term “or” as used herein, means “and/or” unless explicitly indicated to refer to alternatives only or the alternatives are mutually exclusive, although the disclosure supports a definition that refers to only alternatives and “and/or.”

The term “pressure vessel” as used in this specification means a container that is capable of holding a liquid, vapor, or gas at a different pressure than the ambient atmospheric pressure.

The term “polar lipids” or any variation thereof, as used herein, includes, but is not limited to, phospholipids and glycolipids.

The term “reservoir” or any variation thereof, as used herein, includes any body structure capable of retaining fluid. Non-limiting examples of reservoirs include ponds, tanks, lakes, tubs, or other similar structures.

The term “retentate” as used herein may refer to material that remains after the diffusate has passed through a separation device.

The term “solid phase” as used herein refers to a collection of material that is generally more solid than not, and is not intended to mean that all of the material in the phase is solid. Thus, a phase having a substantial amount of solids, while retaining some liquids, is encompassed within the meaning of that term. Meanwhile, the term “liquid phase”, as used herein, refers to a collection of material that is generally more liquid than not, and such collection may include solid materials.

The use of the term “solvent set” as used herein, is used to mean composition comprising one or more solvents. These solvents can be amphipathic (also known as amphiphilic or slightly nonpolar), hydrophilic, or hydrophobic. In some embodiments, these solvents are water miscible and in others, they are immiscible in water. Non-limiting examples of solvents that may be used to practice the methods of the instant invention include methanol, ethanol, isopropanol, acetone, ethyl acetate, and acetonitrile, alkanes (hexane, pentane, heptane, octane), esters (ethyl acetate, butyl acetate), ketones(methyl ethyl ketone (MEK), methyl isobutyl ketone (MIBK)), aromatics (toluene, benzene, cyclohexane, tetrahydrofuran), haloalkanes (chloroform, trichloroethylene), ethers (diethyl ether), and mixtures (diesel, jet fuel, gasoline).

The term “substantially,” as used herein, shall mean mostly.

The term “unsaturated fatty acids” as used herein refers to fatty acids with at least one double carbon bond. Non-limiting examples of unsaturated fatty acids include palmitoleic acid, margaric acid, stearic acid, oleic acid, octadecenoic acid, linoleic acid, gamma-linoleic acid, alpha linoleic acid, arachidic acid, eicosenoic acid, homogamma linoleic acid, arachidonic acid, eicosapenenoic acid, behenic, docosadienoic acid, heneicosapentaenoic, docosatetraenoic acid. Fatty acids having 20 or more carbon atoms in the backbone are generally referred to as “long chain fatty acids”. The fatty acids having 19 or fewer carbon atoms in the backbone are generally referred to as “short chain fatty acids”.

The term “wastewater” as used herein refers to industrial wastewater or municipal wastewater that contain a variety of contaminants or pollutants, including, but not limited to nitrates, phosphates, and heavy metals. “Wastewater” further includes fresh or saline water, effluent from sewage treatment plants and water from facilities in which domestic or industrial sewage or foul water is treated.

The use of the term “wet” as used herein, is used to mean containing about 50% to about 99.9% water content. Water content may be located either intracellularly or extracellularly.

The term “yield” as used in this specification refers to the weight of the recovered material as a fraction or percentage of the estimated dry weight of biomass, even though the sample actually used was never dried. The original dry weight is estimated based on actual dry weights achieved with equivalent samples.

The invention generally encompasses systems and methods for manufacturing algae products and algae derived products that:

a) reduce the net overall carbon footprint of the product;

b) makes the net carbon balance carbon neutral; or

c) carbon negative.

In certain embodiments, the manufacturing process includes two major consecutive parts:

a) using a portion of the algae biomass for the manufacturing of a product.

b) processing the remaining portion of the algae for removal and permanent storage of atmospheric carbon.

In certain embodiments, algae include various components including, but not limited to, carbohydrates, lipids, proteins, and minerals, which enable a broad scope of product manufacturing.

In certain embodiments, the manufacturing of a single or plurality algae products target isolation of one or several classes of compounds (a), while the other compounds (ARB) are used for (b). In general, the overall process of algae product manufacturing includes using one or several classes of algae chemical compounds while using the others for carbon storage to reduce or negate the carbon emissions of the product manufacturing process. In certain embodiments the negative carbon emissions resulting for the manufacturing process used can be sold under the form of carbon credit and represent a type V offset. In certain embodiments, the carbon storage (b) process mimics and enhances the natural carbon mitigation feature of algae where algae residual biomass is returned to the deep sea, and the carbon it contains, initially captured from the atmosphere is stored on a close to permanent time scale.

In certain embodiments, the manufacturing steps include:

(i) obtaining algal biomass;

(ii) the processing of algae into one or several products;

(iii) the recovery of ARB, preferably in the deep sea bed;

(iv) the transformation and shaping of the ARB; and

(v) storage and disposal of ARB.

In certain embodiments, the micro or macro algae are collected from natural habitats, cultivated, or purchased. In certain embodiments, the algae is either fresh or dried. In certain embodiments, the algae biomass is subject to ecofriendly chemical and physical processes to extract one or several algae compounds. In certain embodiments, the algae extracted compound is either:

a) used directly as a product;

b) is further subject to similar biochemical process to produce algae derived final products; or

c) the algae derived compound is further used in a formulation to create an algae derived product.

In certain embodiments, the unused algae compounds (ARB) are recovered to a solid with moisture content ranging from about 1% to about 95%. In certain embodiments, the moisture content of the ARB is about 5, 10, 15, 20, 25, 30, 35, 40, 45, 50, 55, 60, 65, 70, 75, 80, 85, 90, or 95% (w/w); preferably the moisture content is about 75, 80, or 85% (w/v).

In certain embodiments, the ARB is packed using

a) packaging fiber; or

b) binding agent gel to an undefined soft mass shape of volume ranging from about 500 mm³ to about 15 m³.

In certain embodiments, the methods include the use of a drying and compression step as the ARB packing process. In certain embodiments, the packed ARB density when submerged in seawater is superior to the density of the seawater and therefore sinks. In certain embodiments, the pre-dissolution time of the ARB pack is inferior to its sinking time to sea floor. In certain embodiments, the ARB packs are transported from the manufacturing facility to an ocean zone with depth lower than 1500 m and dropped. The ARB packs sank on the ocean floor possess a carbon content between 2-40000 g C/m² and correspond to the amount of carbon stored close to infinite time.

In certain embodiments, the simultaneous use of algae biomass for a product manufacturing and carbon permanent storage results in a reduced, neutral, or negative net balance of carbon emissions of the algae product or algae derived product.

In another embodiment, the invention generally encompasses a method including (1) obtaining algal biomass (2) the processing of the biomass and algae based and derived product manufacturing, (3) the recovery of ARB, (4) the transformation and shaping of the ARB, (5) and storage and disposal of ARB. This technology describes the manufacturing method and processes to reduce the carbon emission of algae or algae derived goods. The overall carbon balance manufacturing of the product is either carbon reduced (i) carbon neutral (ii) and or carbon negative (iii). Since carbon negative product manufacturing processes are the most impactful in the fight on climate change in preferred embodiments, the methods are carbon negative.

In certain embodiments, algae includes marine micro and macroalgae. In certain embodiments, algae biomass is either collected from one or more of the following sources:

Source (1) the natural environment (macroalgae),

Source (2) cultivated, or

Source (3) purchased for a retail market, (which was previously obtained by farmers using strategy (1) or (2)).

In certain embodiments, the algae obtained is either in a dried or fresh, wet form.

In one embodiment, a cultivation system (strategy 2) of algae possessing the lowest CF process involves the collaboration with a fish aquaculture facility using tides instead of pumps. In this system, (also referred as integrated multitrophic aquaculture, ITMA) an algae cultivation tank is placed in the effluent exit of the fish aquaculture system. In such a system, the algae tank receives a high amount of nutrients from the exit aquaculture tanks and water flow allows nutrient fixation by the algae. Such a system is already successfully employed.

In certain embodiments, a controlled cultivation system of algae including aerators and pumps is possible but energy intensive, especially for microalgae cultivation using a tubular and panel system. Using such a strategy will result in a higher carbon footprint of the algae biomass, defeating the overall purpose of this herein technology. In certain embodiments, Source (1), is also an option for sourcing algae with a low carbon footprint, an example of this option is the collection of invasive Sargassum spp. in the Caribbean or Ulva spp. in Britany France. Nevertheless, Source (ii), limits certain compound applications because wild algae accumulate contaminants from the natural habitat.

For any strategy described above, using algal biomass in wet form as a feedstock for the processing is the preferred option. If drying is required, sun-drying instead of using energy intensive dryer class machinery is the second least carbon intensive option available and is the advised methodology for this innovation.

Finally, a location with high annual irradiance will allow higher production yield without having to input energy to the system and is an important factor to take into consideration to obtain efficient marine plant biomass with an overall low CF process.

In one embodiment, the biomass comprises algal biomass. The algal biomass for use in the process of the invention may comprise single-cell micro-algae or macro-algae, and may be harvested from any source, such as bioreactors, aqua-cultured ponds, waste water, lakes, ponds, rivers, and preferably from the seas and oceans.

Non-limiting examples of microalgae that can be used with the methods of the invention include, but are not limited to, Chlorophyta, Cyanophyta (Cyanobacteria), and Heterokontophyta. In certain embodiments, the microalgae used with the methods of the invention are members of one of the following classes: Bacillariophyceae, Eustigmatophyceae, and Chrysophyceae. In certain embodiments, the microalgae used with the methods of the invention are members of one of the following genera: Nannochloropsis, Chlorella, Dunaliella, Scenedesmus, Selenastrum, Oscillatoria, Phormidium, Spirulina, amphora, and Ochromonas.

Non-limiting examples of microalgae species that can be used with the methods of the invention include, but are not limited to, Achnanthes orientalis, Agmenellum spp., Amphiprora hyaline, amphora coffeiformis, amphora coffeiformis var. linea, amphora coffeiformis var. punctata, amphora coffeiformis var. taylori, amphora coffeiformis var. tenuis, amphora delicatissima, amphora delicatissima var. capitata, amphora sp., Anabaena, Ankistrodesmus, Ankistrodesmus falcatus, Boekelovia hooglandii, Borodin ella sp., Botryococcus braunii, Botryococcus sudeticus, Bracteococcus minor, Bracte ococcus medionucleatus, Carteria, Chaetoceros gracilis, Chaetoceros muelleri, Chaet oceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella Lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorellavulgaris, Chlorococcum infusionum, Chlorococcum sp., Chlorogonium, Chr oomonas sp., Chrysosphaera sp., Cricosphaera sp., Crypthecodinium cohnii, Cryptom onas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunali ella primolecta, Dunaliella salina, Dunaliella terricola, Dunaliella tertiolecta, Dunali ella viridi, Dunaliella tertiolecta, Eremosphaera viridis, Eremosphaera sp., Ellipsoid on sp., Euglena spp., Franceia sp., fragilaria crotonensis, fragilaria sp., Gleocapsa sp., Gloeothamnion sp., Haematococcus pluvialis, Hymenomonas sp., 1 sochrysis aff galbana, lsochrysis galbana, Lepocinclis, Micractinium, Micractinium, Monoraphidium minutum, Monoraphidium sp., Nannochloris sp., Nannochloropsis sa lina, Nannochloropsis sp., Navicula acceptata, Navicula biskanterae, Navicula pseud otenelloides, Navicula pelliculosa, Navicula saprophila, Navicula sp., Nephrochloris sp., Nephroselmis sp., Nitschia communis, Nitzschia alexandrine, Nitzschia closteriu m, Nitzschia communis, Nitzschia dissipata, Nitzschia frustulum, Nitzschia Hantzschia na, Nitzschia inconspicua Nitzschia intermedia, Nitzschia microcephala, Nitzschia pu silla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Protothe ca stagnora, Prototheca portoricensis, Prototheca moriformis, Prototheca zopfii, Pse udochlorella aquatica, Pyramimonas sp., Pyrobotrys, Rhodococcus opacus, Sarcinoid chrysophyte, Scenedesmus Armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissf logii, and Viridiella fridericiana.

In one embodiment, the biomass may comprise slurried seaweed. While microalgae may be the fastest growing plants, macroalgae, particularly Macrosystis pyrifera and other members of the Laminariales grow rapidly and grow to very large and readily harvestable sizes, and there are large masses of other seaweed that are in the Phaeophyta such as, but not restricted to, members of the Fucales and the Durvillaeales. Similarly, seaweed from the Chlorophyta, such as Ulva, can also grow rapidly under nutrient rich conditions to give material that is otherwise difficult to get rid of.

In another embodiment, algal biomass comprises micro-algal biomass. Any endemic or cultured microalgae may be used, as a mixed culture or a monoculture, and there are hundreds of thousands of microalgal species. Examples of suitable microalgae include, but are not limited to, microalgae of Division Cyanophyta (cyanobacteria), microalgae of Division Chlorophyta (green algae), microalgae of Division Rhodophyta (red algae), microalgae of the Division Chrysophyta (yellow green and brown-green algae) that includes the Class Bacillariophyceae (diatoms), microalgae of Division Pyrrophyta (dinoflagellates), and microalgae of Division Euglenophyta (euglenoids), and combinations thereof. Examples of Chlorophyta include, but are not limited to, microalgae of the genera Dictyosphaerium, Micractiniumsp, Monoraphidium, Scenedesmus, and Tetraedron, or any two or more thereof. Examples of cyanobacteria include but are not limited to microalgae of the genera Anabena, Aphanizomenon, Aphanocapsa, Merismopedia, Microcystis, Ocillatoria, and Pseudanabaena, or any two or more thereof. Examples of Euglenophyta include but are not limited to Euglena and Phacus. Examples of diatoms include but are not limited to Nitzschia and Cyclotella. Examples of dinoflagellates include but are not limited to Peridinium. In one embodiment, the biomass is cellulose.

In certain embodiment, seaweed in comparison to terrestrial plants possesses the following advantages: (1) they have a higher biomass production rate per unit of area (much faster synthetic rate with a photosynthetic conversion efficiency averaging 3.0% compared to 0.4% for land plants); (2) they do not compete with agricultural plants for arable land; (3) they do not need any input of fertilizer, pesticides or freshwater; (4) they are easier to depolymerize as they contain no lignin in their cellular wall; (5) they have a low harvesting cost; (6) they have a higher scalability; (7) they are not part of the human food chain, (hence do not raise controversy as 1^{st} generation biofuel does about the use of food for energy production); (8) seaweed could be cultured in their natural habitat, which could represent an advantageous contribution for the local ecology; and finally (9), seaweed consume large amounts of CO₂ and act as a carbon sink.

In certain exemplary embodiment, the algae collected for the wild habitat are cleaned, washed with tap water and particulate (1-3 mm), oven dried at 40° C., and stored in a sealed, dry environment for the process of this invention.

In one embodiment macro algae possess production from cultivation for the methods as follow in table 1.

TABLE 1
Seaweed biomass production in several location and system

| | | | Production |
|---|---|---|---|
| | | | (in ton of dry algae · |
| Species | Location | Conditions | ha⁻¹ · ya⁻¹) |
| Ulva | Korea | Ponds culture | 30 |
| | Denmark | Open pond pilot scale - 6 month - little bit of liquid | 45 |
| | | fertilizer - Density: 4 kg · m² | |
| | Italy | Open pond pilot scale - 6 month - little bit of liquid | 65 |
| | | fertilizer | |
| | Florida | −700 L tank oxygen - Rapid water exchange | 74 |
| | | (12 × 700 L · day) - 8 months study - Low density - | |
| | | oxygen - energy intensive | |
| | | −700 L tank No-oxygen - Rapid water exchange | 26.7 |
| | | (12 × 700 L · day) - 8 months study - Low density - no | |
| | | oxygen - non-energy intensive | |
| | Portugal | Small pond - Aeration - Fishery exit - Density: 8 kg · m² | 87.6 |
| General | General | Natural habitat | 16.4 |

In one embodiment macro algae possess production form cultivation for the methods as follow in table 2.

TABLE 2
Carbohydrate's Content for U. lactuca.

| Species | Carbohydrate content |
|---|---|
| Ulva lactuca | 40% |
| | 58% |
| | 24% |
| | 55% |
| | 44% |
| | 53% (rigida) |
| | 43% (Fascista) |
| Average | 45% |

To collect seaweed from the natural habitat (I), it is important to understand the biology of the seaweed. Like terrestrial plants, seaweed bloom seasonally in their natural habitat. For U lactuca, bloom events often occur in South Africa and occasionally in the Yellow Sea. However, the most adequate seaweed for the latter strategy is the brown seaweed Sargassum muticum because it is found in excessive quantities in the Caribbean Sea and abundance has increased in recent years. Sargassum affects 19 country of the Caribbean each year usually from May to September. In such strategy, although there is no need for farm infrastructure and cost, it can be noted that considerable space for drying the collected algae and a storage unit must be included in order to be able to stock for the entire year feedstock needed for the production. Also, the price of the lease of a transportable boat during the blooming months must be considered. Techno-economic assessment reveal that this strategy (I) is estimated at >1.3$ per Kg for a small-scale production.

In certain embodiment, seaweed retail price varies from $400 per ton on Asian retail market to $2000 in the USA. The freight cost estimated at $900/ton from Asia to Europe must also be included. The overall cost of the seaweed for this strategy (II) would be 1.3 $/Kg in the case a pilot plant is based in Europe. While cultivating seaweed, the location of the seaweed farm, and the corresponding leasing price of the land is a strong factor influencing the total price of the seaweed. A collaboration with a fish aquaculture farm could be very advantageous as in exchange for seaweed to feed the fish, the farm would provide the land, the water flow and the nutrients (exiting the fish effluent tank). In recent years, a lot of scientific research has studied the use of algae for the treatment of wastewater. Algae has been shown to strongly enhance the removal of nitrogen, phosphate and chemical oxygen demand indicator on the oxygen available for reaction. Some studies also explored the use of algae for waste of distilleries.

In certain embodiment several systems are possible for the cultivation of seaweed (strategy III), including open sea, raceway ponds and horizontal tubular photobioreactors. Open sea systems are still in the prototype phase. Horizontal tubular photobioreactors are part of a high production system and allows cultivation in an enclosed system that minimizes the risk of contamination, yet the initial cost of installation is high. While growing seaweed, several methods are used to increase its productivity are described in the herein invention. In order to increase seaweed growth, the first method is to use of genetic studies to identify highly productive strains of to be used as base strains for the entire plant. Although this process requires circa three months to enable the reproduction of this individual seaweed to a production scale, this process is greatly recompensated with the consequent increase in biomass productivity during cultivation.

In one embodiment, a cultivation system involves the collaboration with a fish aquaculture facility. Most fish farms are located in estuarine areas and use tides to fill fish tanks. The water that exits the tanks is very rich in nutrients from the fish excrement's and possesses a strong flow (depending on the size of the fish farm). These exit tanks have the ideal conditions for seaweed growth, such as U. lactuca. Implementation of a race pond using the exit water from the fish farm is very beneficial in term of cost and energy demand. In such systems, (1) there is no requirement to buy fertilizer for the seaweed; (2) there is no need to buy pumps and (3) there is no need for a water flow propeller, which all saves energy and money.

A second embodiment of cultivation system involves the classic raceway pond cultivation system where water needs to be pumped from an estuary or the sea to the pond, a propeller is required to create flow for the seaweed to absorb nutrients faster and nutrients are to be added.

In one embodiment the cultivation energetic system of algae is as follow in table 3.

TABLE 3
Estimation of the energy demand for a race way pond system.
ENERGY ANALYSIS
Major equipment operating (as annual average)

| | | | | | Total | | |
|---|---|---|---|---|---|---|---|
| | Power | Time oper | Power consump. | | power consump. | Day | Night |
| Equipment | (kW) | (h · d⁻¹) | (kWh · Yr⁻¹) | Units | (kWh · Yr⁻¹) | (kWh · Yr⁻¹) | (kWh · Yr⁻¹) |
| Seawater pump | 0.2 | 12 | 669.6 | 0 | 310 | 310 | 0 |
| Medium feed | 0.2 | 12 | 669.6 | 0 | 310 | 310 | 0 |
| pump | 0 | 12 | 0 | 0 | 0 | 0 | 0 |
| Sterilization | | | | | | | |
| Process | 0.1 | 12 | 193 | 0.1 | 15 | 15 | 0 |
| Mixing unit | | | | | | | |
| Paddle wheel | 10.4 | 12 | 38654 | 1 | 27068 | 27068 | 0 |
| (day) | | | | | | | |
| Paddle wheel | 10.4 | 12 | 38654 | 1 | 27068 | 0 | 27068 |
| (night) | | | | | | | |
| CO₂ Supply Unit | 0 | 12 | 0 | 0 | 0 | 0 | 0 |
| Harvest Pump | 0.2 | 24 | 1339.2 | 0 | 310 | 155 | 155 |
| Microfiltration | 0.375 | | | | 1085 | 542 | 542 |
| unit | | | | | | | |
| Centrifuge | 4 | 24 | 29760 | 0.0 | 440 | 220 | 220 |
| Temperature | | | | | 0 | 0 | 0 |
| control | | | | | | | |

| | | | |
|---|---|---|---|
| Total power consump. (kWh · Yr⁻¹) | 56606 | 28620 | 27985 |
| Grid supplied electricity (kWh · Yr⁻¹) | 56606 | | |

The biomass used in the processes of the invention may include any type of biomass. For example, marine or freshwater micro algae, marine or fresh water macro algae, seaweed, biomass derived from woody or non-woody land based plants, or combinations thereof. Biomass from woody or non-woody land based plants may include whole crops or waste material including, but not limited to, cellulose, lignocellulose, any grasses (for example, straw), soft wood (for example, sawdust from Pinus radiata), any hard wood (for example, willow), any scrub plant, any cultivated plant, corn, maize, switchgrass, rapeseed, soybean, mustard, palm oil, hemp, willow, jatropha, wheat, sugar beet, sugar cane, miscanthus, sorghum, cassaya, or any combination of any two or more thereof.

In other embodiments, the biomass can be plant material, including but not limited to soy, corn, palm, camelina, jatropha, canola, coconut, peanut, safflower, cottonseed, linseed, sunflower, rice bran, and olive.

The invention also encompasses systems and methods for extracting products including lipids and coproducts (e.g., proteins) of varying polarity from a wet oleaginous material, including for example, an algal biomass. In particular, the methods and systems described herein concern the ability to both extract and fractionate the algae components by doing sequential extractions with a hydrophilic solvent/water mixture that becomes progressively less polar (i.e., water in solvent/water ratio is progressively reduced as one proceed from one extraction step to the next). In certain embodiments, the interstitial solvent in the algae (75% of its weight) is initially water and is replaced by the slightly nonpolar solvent gradually to the azeotrope of the organic solvent. This results in the extraction of components soluble at the polarity developed at each step, thereby leading to simultaneous fractionation of the extracted components. Extraction of proteinaceous byproducts by acid leaching and/or alkaline extraction is also encompassed by the claimed invention. Proteins extraction methods that are ecofriendly such aqueous extraction, enzymatic assisted extraction, osmotic shock, spray drying, air classification, hot water buffer, subcritical water extraction, pulse electric field, accelerated extraction, alkali and precipitation amongst other methods are preferred.

Another embodiment of the methods and systems described herein involves varying the ratio of algal biomass to solvent based on the components to be extracted. In one embodiment, an algal biomass is mixed with an equal weight of solvent. In another embodiment, an algal biomass is mixed with a lesser weight of solvent. In yet another embodiment, an algal biomass is mixed with a greater weight of solvent. In some embodiments, the amount of solvent mixed with an algal biomass is calculated based on the solvent to be used and the desired polarity of the algal biomass/solvent mixture. In still other embodiments, the algal mass is extracted in several steps. In an exemplary embodiment, an algal biomass is sequentially extracted, first with about 50-60% of its weight with a slightly nonpolar, water miscible solvent. Second, the remaining algal solids are extracted using about 70% of the solids' weight in solvent. A third extraction is then performed using about 90% of the solid's weight in solvent.

Each algae species possesses its characteristic trait and certain species are preferred accordingly for the extraction of a certain compounds, e.g., Helium spp and Gracilaria spp give the most qualitative agar; species containing high and easily recoverable glucose such as Laminaria, Gracilaria and Ulva spp are usually preferred to produce bioethanol or the production of PHAs. Brown species usually possess higher amounts of antioxidants (which is the contrary for red and green which do not possess toxic compound for humans) and are primarily used for the cosmetic industry.

Table 4 reports a list of compounds that can be extracted from algal biomass but does not limit to this list alone. Some of the most common compounds are polysaccharides, pigments, lipids, fatty acids, proteins, amino acids, vitamins and minerals.

TABLE 4
Algae bioactive compounds, their properties, and applications.

| | | | | Industry | |
|---|---|---|---|---|---|
| Chemical group | | Compounds | Properties | application | Products |
| Polysaccharides | | Alginate | Anti-viral | Fermentation, | Facial cream, |
| | | Cellulose | Anti-tumor | biomaterials, | hair gel, |
| | | Fucoidan | Anti-inflammatoiy | food, | Thickeners, |
| | | laminarin | Anti-oxidative | agriculture, | emulsifiers |
| | | Agar | Anti-bacterial | pharmaceuticals, | Biodiesel |
| | | Carrageenan | | cosmetics, | Biomethane |
| | | Cellulose | | cosmeceuticals, | Acetone |
| | | Furcellaran | | and functional | Bioethanol |
| | | Mannan | | food, biofuel | Perfume, |
| | | Porphyran | | production | fragrances, |
| | | Xylan | | | Ready to make |
| | | Amylose | | | beverage, |
| | | Amylopectin | | | Hand sanitizer |
| | | Inulin | | | Premium vodka |
| | | Pectin | | | gin |
| | | | | | PHAs |
| | | | | | Paper |
| Pigments | Phycobilin | Phycocyanin | Anti-fungal | Pharmaceuticals, | |
| | | Phycoerythrin | Anti-bacterial | fluorescent dyes | |
| | Carotenoids | Lycopene | Antiviral | Food, animal | Ink, facial cream, |
| | | Astaxanthin | Anti-inflammatoiy | feed, cosmetics, | anti- aging |
| | | Fucoxantina | Antitumor | pharmaceuticals, | cream |
| | | Zeaxanthin | | nutraceuticals | |
| | | Lutein | Anti-oxidative | | |
| | | Astaxanthin | Anti-fungal | | |
| | | Fucoxanthin | Anti-bacterial | | |
| | | Zeaxanthin | | | |
| | | Lutein | | | |
| | Chlorophylls | Chlorophyll a | Anti-fungal | Food, feed, | Food colorant, |
| | | Chlorophyll b | Anti-bacterial | cosmetics, | facial cream, |
| | | Chlorophyll c | | pharmaceuticals | anti- aging |
| | | | | | cream |
| Lipids and fatty acids | | Polyunsaturated FA | Anti-inflammatory | Food, animal | Supplement |
| | | Monounsaturated FA | Anti-oxidative | feed, cosmetics, | pills |
| | | Unsaturated FA | Anti-fungal | pharmaceuticals, | |
| | | Cholesterol | Anti-bacterial | biofuel, | |
| | | Fecosterol | | nutraceuticals, | |
| | | Ergosterol | | functional food | |
| | | 24-methylenecholesterol | | | |
| | | Sterols | | | |
| | | Fecosterol's | | | |
| | | PUFAs | | | |
| Minerals | | K, Ca, Mg, Na, Zn, Co, | | Fertilizers | |
| | | Cu, I, B | | | |
| micronutrients | Polyphenols | Phlorotannin | Antioxidant | Food, | facial cream, |
| antioxidant | | Catechin | Anti-viral | cosmetics, | anti- aging |
| | | Phenolic acid | Anti-tumor | pharmaceuticals | cream |
| | | Flavonoids | Anti-inflammatory | | |
| | | Tannins | Anti-fungal | | |
| | | Lignans | Anti-bacterial | | |

| | | | | |
|---|---|---|---|---|
| Proteins and amino acids | Mycosporine like amino | Anti-viral | Food, feed, | Vegetarian burger, |
| | acid | Anti- | pharmaceuticals | fish feed, |
| | Lectins | inflammatory | | mollusk feed |
| | Cytokinin | Anti-oxidative | | |
| | Auxins | Anti-bacterial | | |
| | Gibberellins | | | |
| | Abscisic acid | | | |
| | Ethylene | | | |
| | Histidine | | | |
| Vitamins | B₁₂, K, C, E, A, D | Anti-oxidative | | Pills supplement |
| Diterpenes | Dolabellones | | | |
| | Hydrazulenoids | | | |
| | Xenicanes | | | |
| | Extended Sesquiterpenoids | | | |

In this invention, a certain portion, usually a single specific algal compound (i.e., glucans for glucose recovery and transformation or protein for food additives) are extracted and used for consumer product manufacturing, while the recovered algal residual biomass is reintroduced to its natural ocean end of life cycle where it can accomplish its role as a blue carbon mitigator. It is possible to target several chemical groups of algae compounds in a cascade system.

Algal biomass contains 25% carbon content on average in its natural habitat [4] and circa 30% for cultivated algae. Each algal compound possesses carbon which varies depending on its general chemical formula, season, habitat, nutrients availability, plant stress and other factors. Table 5 shows the repartition of the carbon in an algae blend composed of 70% Ulva lactuca and 30% sargassum muticum. In terms of carbon repartition, the carbon of each algal compounds targeted for a particular product or application, is transposed in the product, while the carbon of the ARB corresponds to the sum of all the other carbon compounds. It can be noted that while extracting one compound, whichever this compound is, a high amount of carbon is left in the remaining compounds and supports the broad application potential of this herein technology.

TABLE 5
Main algae compound weights in an algae blend of 70% Ulva lactuca and 30% Sargassum muticum,
the carbon quantity and percentage of each compound, the quantity of carbon remaining in after extraction
of the relative carbon compound and the equivalent of this carbon in C0₂.

| | | | | Quantity of | carbon | Quantity of |
|---|---|---|---|---|---|---|
| | Quantity of | Quantity of | Percentage | carbon remaining | remaining | CO₂ eq |
| | the Seaweed | Carbon in | of the | after extraction | after | relative to |
| | blend | each algae | compound | of the algae | extraction of | the total |
| | compound | compound | carbon | compound | the algae | C_{ARB} mass * |
| Main Algae | (in g/100 g | (in g/100 g | content | (in g/100 g | compound | (in g CO₂/ |
| compounds | dry algae) | dry algae) | (in %) | dry algae) | (in %) | 100 g dry algae) |
| Polysaccharides | 64.0 | 23.7 | 37.1 | 8.5 | 26.3 | 31.1 |
| Lipids | 0.5 | 0.2 | 48.6 | 32.0 | 99.3 | 117.4 |
| Proteins | 15.0 | 8.2 | 54.9 | 24.0 | 74.4 | 88.0 |
| Ashes | 20.5 | 0.0 | 0.0 | 32.2 | 100.0 | 118.2 |
| Total | 100 | 32 | — | — | — | — |

* NB: It is assumed that the process allows 100% recovery of ARB.

In various embodiments, algae compounds can be subject to several manufacturing strategies to create a desired product. Algae products or algae derived products follow the following manufacturing steps:

Process A: (I) Extraction; (II) recovery delivery of final product. Exemplary products produced by this process include, but are not limited to, agar, antioxidant, proteins, minerals etc.

Process B: (I) Extraction; (II) recovery; and (III) targeted compound used in formulation with a plurality of compounds and delivery of final product. Exemplary products produced by this process include, but are not limited to, antioxidant to be used in anti-aging cream, protein for vegetable burgers, agar, personal lubricant.

Process C: (I) Extraction; (II) recovery (optional); (III) targeted compounds subjected to further biochemical process; (IV) recovery of algae derived compound and delivery of final product. Exemplary products produced by this process include, but are not limited to, the monosaccharide of algae can be used as feedstock for microorganism such as yeast to produce ethanol or bacteria to synthesize PHAs.

Process D: (I) Extraction; (II) recovery (optional); (III) targeted compounds subjected to further biochemical process; (IV) recovery of algae derived compound; (V) targeted compound used in formulation with a plurality of compounds; and delivery of final product. Exemplary products produced by this process include, but are not limited to, fragrances, hand sanitizer, ready to drink alcoholic beverage.

In various embodiments, all algae products and algae derived product manufacturing processes (Processes A to D, supra) apply to this invention. Since the ARB is released into the environment, the steps Process A: (I), (II); Process B: (I), (II); Process C: (I), (II), (III); and Process D: (I), (II), (III), (IV) preferably do not:

(i) use hazardous chemicals such as acid treatment for biomass hydrolysis as the use of such hazardous chemical end up in the ARB; and/or

(ii) generate contaminants such as PCBs dioxins, mycotoxins, heavy metals (as described by the JRC in the Potential chemical contaminants in the marine environment; the EPA in the National Recommended Water Quality Criteria, or similar country legislation), unless a removal of the above chemical process step is added and later verified by ecotoxicological assessment.

The addition of steps results in increased energy usage and CO2 equivalent emissions. The use of eco-friendly algae compound extraction methods is preferred and recommended to maximize the purpose of the herein invention. The main green process methods are hydrothermal pretreatment of the biomass (water and heat) to break down the cellular wall and release of intracellular material; and enzymatic treatment to further degrade the algal polymers and extract target compounds.

In some embodiments of the invention, a single solvent and water are used to extract and fractionate components present in an algal biomass material. In other embodiments, a solvent set and water are used to extract and fractionate components present in an algal biomass material. In some embodiments the algal biomass material is wet. In other embodiments, the algal biomass material is algae.

In certain embodiments, the extraction process results in over 15%, 20%, 25%, 30%, 35%, 40%, 45%, 50%, 60%, 70%, 80%, 85%, 90%, 92%, 94%, 96%, 98%, or 99% recovery. In certain embodiments, the small amount of polar lipids in the remaining biomass enhances its value when the remaining biomass is used for feed. This is due, at least in part, to the high long chain unsaturated fatty acid content of the biomass. In addition, ethanol extracts can further be directly transesterified. Furthermore, unlike the existing conventional methods, the methods and systems described herein are generic for any algae and enable recovery of a significant portion of the valuable components, including polar lipids, in the algae by, for example, the use of a water miscible organic solvent gradient.

In various embodiments, the systems and methods disclosed herein can start with wet algal biomass, reducing the drying and dewatering costs. Compared to conventional extraction processes, the disclosed extraction and fractionation processes should have relatively low operating costs due to the moderate temperature and pressure conditions, along with the solvent recycle. Furthermore, conventional extraction processes are cost prohibitive and cannot meet the demand of the market.

In certain embodiments, the biomass is fed as a slurry in a fluid, the fluid is preferably water. The amount of water must be sufficient to permit the slurry to be pumped or otherwise moved. The amount of water must also be sufficient to ensure an adequate volume of fluid phase in a subcritical reaction, or to maintain the required pressure in a supercritical reaction. Therefore the amount of water required depends on the final temperature and pressure used, the pumping equipment used, and the reactor configuration. It will also depend on the nature of the biomass, as some biomass, particularly dried biomass, absorbs water.

The more dilute the biomass slurry, the more energy is wasted heating water. Therefore, higher concentrations of algae are desirable. In the case of microalgae, the lower practical concentration of microalgae is about 1 to 2% by weight. From an operational point of view, for a continuous flow process about 50% by weight of microalgae is an upper limit and about 80% by weight for a batch reactor with proper allowance for headspace. The actual concentration used will be influenced by cost, including the cost of concentrating the microalgae prior to use in this invention, and this invention applies to all such concentrations. A person of ordinary skill in the art will be able to select the appropriate biomass concentration having regard to that skill

Biomass from different sources may be mixed, e.g., cellulosic material with microalgae. Biomass may have been pretreated, e.g., chemical pretreatment, hydrolysis, size reduction, etc.

In various embodiments, the biomass concentration of the slurry comprises about 1, 2, 3, 4, 5, 6, 7, 8, 9, 10, 15, 20, 25, 30, 35, 40, 45, 50, 55, 60, 65, 70, 75 or 80% by weight and useful ranges may be selected between any of these values (for example, about 1 to about 10, about 1 to about 20, about 1 to about 30, about 1 to about 40, about 1 to about 50, about 1 to about 60, about 1 to about 70, about 1 to about 80, about 10 to about 30, about 10 to about 40, about 10 to about 50, about 10 to about 60, about 10 to about 70, or about 10 to about 80% by weight).

In certain embodiments, the algal biomass/aqueous slurry may be heated at any temperature of about 150° C. to about 500° C. under pressure, including at least about 150, 160, 170, 180, 190, 200, 210, 220, 230, 20, 250, 260, 270, 280, 290, 300, 310, 320, 330, 340, 350, 360, 370, 374, 380, 390, 400, 410, 420, 430, 440, 450, 460, 470, 480, 490 or 500 oC, and useful ranges may be selected between any of these values (for example, about 200 to about 450, about 300 to 380, about 340 to about 380, and about 370 to about 450° C.).

In one embodiment the algal biomass is heated at a temperature of from about 300° C. to about 375° C. preferably from about 350° C. to about 375° C. if the reaction is intended to take place under subcritical conditions. In one embodiment the method of the invention is for the manufacture of aromatic compounds at subcritical temperatures.

In another embodiment the biomass is heated at a temperature of from about 375° C. to about 450° C. if the reaction is intended to take place under supercritical conditions.

The algal biomass may be heated for a time period of about 0.5 seconds to about 12 hours. In various embodiments the biomass may be heated for about 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650 or 700 minutes, and useful ranges may be selected between these values (for example, about 1 to about 60, about 1 to about 120, about 1 to about 180, about 1 to about 240 minutes, about 5 to about 60, about 5 to about 120, about 5 to about 180, or about 5 to about 240 minutes).

In one embodiment the time period is preferably about 5 minutes to about 3 hours or about 5 minutes to about 60 minutes. As a general principle, the lower the chosen temperature, the longer the heating time required to achieve a specific objective. Accordingly, the heating time can be selected for convenience. The overall yield increases progressively with time, at least to 30 minutes, with the greatest increase up to 10 minutes, but the increased yield after 10 minutes sometimes arises through the formation or extraction of higher molecular weight products, hence shorter times may be more desirable if the volatile components are more desired.

In one exemplary embodiment, the autohydrolysis was carried out in a Versoclave Buchiglasuster of 1600 L capacity stainless steel Pressure reactor (Versoclave Buchiglasuster, Switzerland). The system possesses a water flow system in order to cool the installation. Water and dried Ulva lactuca are mixed at a concentration 12 kg of water per 1 kg of algae (liquid to solid ratio of 12 g/g, equivalent to a consistency of 7.69% and or finally to a substrate concentration of 7.69% w/v). With an experimental loading in the reactor of 800 kg total weight, the mixture was stirred at 50 rpm using the ATEX stirrer drive of the pressure reactor and hydrothermally treated to reach several maximum temperatures (Tmax) and then cooled to room temperature. The harshness of the pretreatment was expressed with the use of some mathematical equation known as the severity factor of the treatment. Using the formula:

$${S}_{0} = log {R}_{0} = log ( {R}_{{0}_{HEATING}} + {R}_{{0}_{COOLING}} ) = log [ {{\int }_{0}}^{t MAX} exp ( \frac{T ( t ) - {T}_{REF}}{ω} ) \cdot dt ] + [ {{\int }^{tF}}_{t MAX} exp ( \frac{{T}^{′} ( t ) - {T}_{REF}}{ω} ) \cdot dt ]$$

where R₀ is the severity factor (min), t_{MAX} (min) is the time employed to achieve the target temperature t_{MAX} (° C.), t_{F(min)} is the time used for the whole heating-cooling period, and T₍ₜ₎ and T₀₍ₜ₎ represent the temperature profiles in the heating and cooling stages, respectively. Specifically, T₍ₜ₎ and T₍ₜ₎ represent the variation of the dependent variable; Temperature (T) with the independent variable time (t). There is no equation to represent this variation of T with t so, instead, the temperature profile of heating and cooling stages are recorded in each experiment, as a set of (temperature, time) points. These two sets of points are used for the numerical resolution of the two integrals, using the Simpon's Rule. Calculations are made using the values 14.75° C. and 100° C. for w and TREF.

At a given temperature, the relative product distribution is a function of time, therefore there is no optimum reaction time other than in terms of which are the desired products. As a general rule, longer times favor condensation reactions, hence the products are less volatile, more viscous, and generally are more likely to require significant downstream processing. Shorter times may be preferred, even if total processing of microalgae is not achieved, in order to maximize the production of higher value products.

In another embodiment, the algal biomass is heated for a first period at a first temperature for about 1 to about 120 minutes or more and for a second period at a second temperature for about 1 to 120 minutes or more, where each of the first and second periods may be independently selected from at least about 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120 minutes, and useful ranges may be selected between these values (for example, about 1 to about 15, about 1 to about 30, about 1 to about 45, about 1 to about 60, about 1 to about 75, about 1 to about 90 or about 1 to about 105 minutes). In various embodiments the first temperature is about 150 to about 373° C., about 300 to about 373° C. or about 340 to about 373° C. In various embodiments the second temperature is about 374 to about 500 or about 374 to about 450° C.

The pressure generated is dependent on the amount of water present, as the water provides the pressure. There must be sufficient water present to provide a liquid phase, and the appropriate water partial pressure if supercritical, as otherwise excessive charring may occur.

Additional pressure may be applied to achieve certain objectives, e.g. increasing the pressure generally increases the yield of aromatic products.

In various embodiments, the pressure vessel which may be utilized for the processes of the present invention may be a tank, a batch reactor, a continuous reactor, a semi-continuous reactor of stirred-tank type or of continuous staged reactor-horizontal type or vertical-type, or alternatively of a tubular-type or tower-type reactor. A fluidized-bed or slurry-phase reactor may also be employed. Such vessels and reactors may be further specified as appropriate for use with the type or phase of catalysts and/or reagents which may be used.

Accordingly, in one embodiment the aqueous slurry is heated under autogenous pressure in the pressure vessel. In various embodiments the pressure in the pressure vessel is about 1, 2, 3, 4, 5, 6, 7, 8, 9, 10, 15, 20, 25, 30 or 35 MPa and useful ranges may be selected between any of these values (for example, about 1 to about 30, about 5 to about 25, or about 10 to about 25 MPa).

In one embodiment, non-isothermal autohydrolysis pretreatment is used for the biomass because, although it is still energy demanding, the pretreatment duration is relatively short and only uses water as a reagent in comparison to other methods such as acid, which generate industrial chemical hazards and waste on top of the energy required to catalyze its reaction and make it shorter. This step of the process is therefore amongst one of the greenest methods, especially if the reactor employs biochar (co-firing system) from algae post fermentation residual or solar energy.

In a certain embodiment, all pretreated biomass released high percentage of glucan in the pretreated biomass (section 3.2) as compare to the initial percentage of glucan in the non-pretreated biomass (section 3.1) and because all of the pretreated biomass experiments resulted in 100% glucan to glucose conversion (section 3.4), it can be concluded that pretreatment of seaweed does enable a higher enzymatic susceptibility for its hydrolysis, which enables higher hydrolysis into monosaccharide such as glucose. This is particularly true for the biomass pretreated at S₀=2.64 (Tₘₐₓ=155° C.), which resulted in the highest percentage of glucan in the composition of its solid phase (section 3.2) and S0=2.60 (Tmax=150° C.) that allowed the most transformation of glucan to glucose.

In another embodiment, a catalyst is added to the algal biomass prior to heating. For example, phosphate is added and may be either soluble or insoluble in water and may be added as a specific phosphate, such as trisodium phosphate, or it may be formed in situ. For example, an ammonium phosphate may be formed by adding phosphoric acid, then adding sufficient ammonia to form the desired phosphate anion, which can be monitored by measuring the pH. Thus if using ammonium dihydrogen phosphate, ammonia would be added until the pH was approximately 6. Calcium dihydrogen phosphate could be prepared either by adding calcium hydroxide to phosphoric acid, or any acid to calcium phosphate until the pH is approximately 6. Similarly, an insoluble phosphate could be prepared by adding a soluble phosphate and a suitable counterion, and adjusting the pH. For example, if phosphoric acid is added, followed by sufficient slaked lime, or a soluble calcium salt, followed by any alkali, a precipitate is obtained with pH>7. For the purposes of this invention, the precipitate so obtained will be termed calcium phosphate, although in practice it may well be calcium hydroxyapatite. Suitable cations to act as counterion for the phosphate species may comprise monovalent cations including but not restricted to sodium, potassium, ammonium or hydrogen; divalent cations including but not restricted to magnesium, calcium, strontium, barium, zinc, copper, nickel, ferrous, manganous; or trivalent cations including but not restricted to aluminum, chromic and ferric; or any combination of any two or more thereof.

The exact choice of counterion, or the method of adding it, is dependent on the desired products to be made, such possible variation being illustrated by example. The nature of the products are also dependent on the amount of catalyst, at least in some cases, hence the amount of catalyst used, or which catalyst is used, may be influenced by the demand for given products at the time.

The presence of a catalyst influences the composition of organic chemical products obtained in methods of the invention. Depending on the temperatures and heating times used, the process will produce a range of organic chemical products that may be useful without further purification, or more likely will be further separated into chemical families or individual compounds for other uses.

In one embodiment the aqueous slurry comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of one or more catalysts and useful ranges may be selected between any of these values (for example, about 1 to about 5, about 1 to about 10, about 1 to about 15, about 1 to about 20 or about 1 to about 30% by weight).

Optionally, before separating one or more organic chemical products, the mixture resulting from the heat and pressure treatment step may be filtered to recover solids, including catalyst or reagent materials.

In other embodiments, one or more organic chemical products can be separated from the mixture resulting from the heat and pressure treatment step by any means known in the art including decanting the organic fraction off the aqueous fraction or extracting the fractions with one or more organic solvents. Options for extracting the aqueous fraction and organic fraction are described below.

In another embodiment, the aqueous fraction or the algal biomass residue may be extracted with one or more organic solvents to obtain organic material adhering to the biomass residue or forming a colloidal distribution in the aqueous fraction or dissolved in the aqueous fraction.

In another embodiment, extraction may be carried out using any organic solvent or combination of organic solvents that are insoluble in water. Examples include, but are not restricted to, light hydrocarbons such as light petroleum spirit, pentane, methylene chloride and other halogenated hydrocarbons, toluene and other aromatic hydrocarbons, ethyl acetate and other esters, diethyl ether and other ethers, and also materials such as propane and butane that are gases at normal temperatures but can be liquids under suitable pressure if such pressure was applied.

In another embodiment, the organic fraction may be simply separated from the aqueous fraction to provide an organic chemical product that may be used as a fuel precursor. In another embodiment the organic fraction may be further separated into one or more organic chemical products that may be useful in applications including but not limited to biofuel production or providing feedstock for other chemical processes.

In another embodiment, the further separation step may be a distillation step, either single or multistage, or by flashing and condensing volatiles from the reaction. The distillation step may be prior to extraction, in which case water is also distilled. Various fractions obtained by extraction or partitioning may also be distilled.

Separation of organic chemical products can be achieved by acidifying or alkalizing the aqueous fraction prior to extraction into an organic solvent, or by extracting a solution of organics in an organic solvent with aqueous acid or alkali. For example, acidification of the aqueous fraction will protonate any organic acids present, allowing them to be subsequently extracted from the aqueous fraction with organic solvents. In one embodiment the aqueous fraction is alkalized and the resulting alkaline aqueous fraction is extracted with organic solvent to produce nitrogen bases. Acidification and basification may be carried out in any order.

Organic chemical products can also be obtained from the aqueous fraction. If the aqueous fraction is acidified and extracted with solvent such as pentane or methylene chloride carboxylic acids are obtained, mainly acetic, propionic, and methylated butyric and valeric acids. These products are likely obtained by deamination of amino acids, and some lipid acids, including palmitic and oleic acids. Phenol and cresol were also found, probably because the ammonia solutions were of a pH suitable for partial dissociation. Lactams such as 2-pyrrolidinone may also dissolve in the aqueous fraction, in which case they may cause some organic materials such as aromatic hydrocarbons to accompany them.

If the aqueous fraction is made basic, extraction will obtain organic bases. Since some bases include piperidine, the pH should be raised to 12 to extract this material. Lactams will also be extracted with the organic bases.

The order of pH variation is not critical, and the first extraction may be carried out at either high or low pH, or if desired, at an intermediate pH to gain a particular separation. For example, an initial extraction at pH 7 would lead to a fraction containing hydrocarbons, pyrazines, lactams, etc, but leave both carboxylic acids and saturated amines in solution.

In various embodiments, the pH is about 1, 2, 3, 4, 5, 6, 7, 8, 9, 10, 11, or 12.

Changing the pH of the organic fraction may allow some organic chemical products present in the organic fraction to be able to be extracted into an aqueous solution. For example, if an acid solution that has been extracted is then made alkaline, diazines and similar organic chemical products can be extracted using organic solvents.

A higher yield of volatiles is often achieved at supercritical conditions, however there is often significant difference in the nature of the products, hence subcritical conditions may be desirable for specific products.

The organic chemical products obtained varied somewhat depending on the nature of the phosphate, but there were a number of products occurring at levels of approximately 2% that did not appear to change significantly with the phosphate catalyst used. One such compound was indole, and there were a number of higher boiling materials that appeared to contain pyrrole or pyrazine rings.

Certain fractions or chemical compounds may be separated and used as such, while the residue, which includes the higher boiling fraction, may be hydrocracked or otherwise treated by methods known to those practiced in the art to convert them to more conventional fuels.

The organic chemical products obtained by the process of the invention may be separated into their chemical components using known purification techniques. These products can be used in many applications, including as a chemical feed stock for the synthesis of other chemicals. For example, the pyrazines can be used as flavor additives in the food industry, indoles may be used in the perfume industry, while the lactams have many uses, including as intermediates for polyamides or for inclusion as an amide in a condensation polymer, or as high boiling polar solvents. Amides produced by the process of the invention may be useful for subsequent conversion into solvents such as acetonitrile, or into surfactants and cationic detergents.

The separated chemical components produced by the process of the invention may also be used as chemical intermediates for the production of biopolymers. The use of lactams to make polyamides has been noted, but the oxidation of 2,5-dimethylpyrazine makes available a useful diacid, which may be a component of polyamides, while diols produced by the reaction with macroalgae may have value in polyesters. Such polymers, with high levels of nitrogen or oxygen may have particularly useful properties in terms of interaction with water and polar molecules that are difficult to get otherwise.

In embodiments of the invention, the algal biomass can have a variety of components. These components may include, but are not limited to:

(a) Lipids, including straight chain hydrocarbon fatty acids, including those bound in triglycerides, phospholipids, glycolipids and lipoproteins.

(b) Hydrocarbon-based components including terpenes and related materials, such as steroids and steroid precursors, etc are also found in marine algae, and while in land plants these are frequently oxygenated, in marine plants they may also form sulfides or halogenated (usually brominated) species.

(c) Proteins including various polymers based on 21 amino acids that form a number of polymers with a large variety of properties simply through the variation in the ways they are combined.

(d) Nucleic acids including polymers based on phosphate diesters of ribose or 2-deoxyribose, which are substituted through C-1 with nucleotides.

(e) Carbohydrates including sugars, which can include those found as polymers.

(f) Phenolics including polyphenols, such as lignans, tannins, etc.

(g) Miscellaneous functional materials, such as chlorophylls.

(h) Water, as a main component of biomass, the presence of water affects its processing as it requires a large amount of energy to remove it.

In various embodiments, the manufacturing of the best quality Agar is carried out with red algae genus, principally Gracilaria spp and Gelidium spp but is not limited to these species. In various embodiments, agar and carrageenan are extracted by alkali treatment, microwave assisted extraction and sound assisted extraction amongst other techniques. The extraction process is followed by filtration, gelation, freezing, thawing, bleaching dialysis, syneresis, drying and milling.

In various embodiments, methods to produce paper from seaweed use the entire algae biomass combined with wood pulp and hence do not generate ARB, some only require the cellulose of the seaweed and therefore generate potentially storable ARB. In certain embodiments, a method of paper manufacturing uses a colloid mill for grinding the algal biomass and vibrating screen to filter larger particles. In certain embodiments, mixtures of calcium carbonate, a diketenic-type synthetic glue, cationic starch are usually added in a paper refiner to produce paper.

In certain embodiments, algal saccharides extraction for microorganism culture and derived product manufacturing includes polyhydroxyalkanoates (PHAs) and bioethanol

In various embodiments, the methods of sugar recovery include, but are not limited to, either one or a combination of the following treatments dry heat, aqueous solution, microwaves, oxidative and hydrothermal, air classification, subcritical water, enzymatic assisted extraction, fungal hydrolysis, alkali, acid (if recovered by dehydration or concentration). The hydrolysate medium containing sugars is used as an energy medium for the culture of microorganisms to produce a compound (i.e., an algae derived compound). Ethanol is produced via fermentation using microorganisms such as, for example, the yeast S. cerevisiae. The ethanol contained in the ferment is recovered either by distillation, membrane separation such as pervaporation, or a hybrid system to obtain hydrous or anhydrous ethanol. Algae hydrolysate containing sugars is used as feeding medium for the growth of H. boliviensis and H. elongata to synthesize PHAs.

In various embodiments, one of ordinary skill will recognize that the fermentation can be achieved with or without yeast. In certain embodiments, exemplary, non-limiting examples of yeasts that can be used to obtain various alcohols are illustrated in Table 6a:

TABLE 6a

| Beverage | Yeast Involved | Comments |
|---|---|---|
| [symbol] | [symbol] | [symbol] |
| Wine | [symbol] | [symbol] |
| Whisky | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

In other embodiments, exemplary, non-limiting examples of yeasts and the fermentation conditions to obtain various alcohols are illustrated in Table 6 b:

TABLE 6 b

| Yeast/Bacterial strain and | |
|---|---|
| Fermentation process | Conditions |
| Brettanomyces custersii KCCM 11490 | pH 4.8-5.5, 27-30° C. |
| E. coli | pH 7, 170 rpm, 37 C. for 12 h |
| E. coli KO11 | 30° C. for 24 h |
| Ethanol Red yeast | 32° C. |
| Pichia angophorae KCTC 17574 | 5% 30° C. at 200 rpm, 136 h |
| Pichia angophorae Zymobacter palmae | pH 6, 30° C. |
| Pichia stipitis | pH 5, 200 rpm |
| Saccharomyces cerevisiae | 109 CFU/ml 28° C., 120 rpm |
| | for 48 h |
| Saccharomyces cerevisiae MTCC180 | 28° C. for 12 h |
| Saccharomyces cerevisiae IAM4178 | 30° C. for 36 h |
| Saccharomyces cerevisiae ATCC24858 | pH 5.5, 150 rpm, 30° C. |
| | for 24 h |
| Saccharomyces cerevisiae KCTC 1126 | pH 5.5, 30° C., 220 rpm |
| | for 12 h |
| Saccharomyces cerevisiae KCTC 1126 | pH 5, 30° C., 150 rpm for 114 h |
| Saccharomyces cerevisiae NCIM | 5% v/v, 30° C., 150 rpm, |
| | pH 6.4-6.8 for 48 h |
| Scheffersomyces stipitis | pH 5.5, 30° C., 200 rpm |

In certain embodiment the hemi-cellulose structure of the biomass cellular wall (complex polysaccharides) are broken down (1), as well as (2) the released cellular material into much simpler chains of polysaccharides, and further into more simple sugars or monosaccharides. Those monosaccharides are fermentable sugars that are then consumed by a yeast, which generates ethanol as by product: this is the fermentation process. Saccharomyces cerevisiae is the most common yeast used for the conversion of hexoses such as glucose and galactose to ethanol. As cellulosic hydrolysate generates fermentation inhibitors, the use of industrial strains that have been selected as more resistant and more productive than common laboratory strains of yeast, are hence preferred for bioethanol production.

In certain embodiment the ethanol yield obtains range from 0.078 to 0.273 g EtOH/g dry algae as showed in Table 7.

TABLE 7
Resuming the average of the ethanol yield from the
transformation of seaweed into bioethanol. Calculated
with the ethanol density at 20° C. of 0.7851.

| Bioethanol yield | Unit |
|---|---|
| 0.097 | g EtOH/g dry algae |
| 0.12 | g EtOH/g dry algae |
| 0.141 | g EtOH/g dry algae |
| 0.273 | g EtOH/g dry algae |
| 0.078 | g EtOH/g dry algae |
| 0.06 | g EtOH/g dry algae |
| 0.128 | g EtOH/g dry algae |
| 0.163* | mL/g of dry algae |
| 163 | liters per ton of dry algae |

*Calculated with the ethanol density at 20° C. of 0.7851

In one exemplary embodiment, one industrial Saccharomyces cerevisiae strain: Ethanol Red® is used for the fermentation assays. The stock culture was kept on YPD (1% (w/v) of yeast extract, 2% (w/v) of bacto-pectone and 2% (w/v) of glucose) agar at 4° C. In the inoculation step, yeast strains are grown in Erlenmeyer flasks containing 10 g yeast extract/L, 20 g peptone/L, and 20 g glucose/L for 15 h at 30° C. Inoculum media is centrifuged for 10 min at 4000 rpm and 4° C. in order to collect the cells which were resuspended in 0.9% NaCl to a concentration of 200 g fresh yeast/L. The SSF experiments are inoculated with 8 g of this suspension/L (corresponding to 1.5 g dry cell/L).

In certain embodiment, the breakdown of the biomass into simple sugars (hexoses), two distinguishable methods are employed: one use enzymes (proteins), the other uses acids (usually sulfuric or phosphoric acid which are later recovered by dehydration). Acids have shown to be efficient but represent a chemical hazard, while enzymes pose as much greener approach, but the price represents an important factor in the overall biorefinery process.

In certain embodiments, the use of a pretreatment before application of enzymes allows a higher yield of sugar and hence of ethanol. Extensive research has been carried out from dry heat, to aqueous solution, to microwaves, acid base, oxidative and hydrothermal.

In certain embodiment the use of a simultaneous saccharification and fermentation (SSF) is often employed to reduce the overall duration of the process. In this system, while the enzymatic hydrolysis releases sugar, the sugar is directly consumed by the yeast, resulting in only a two-step overall process to produce bioethanol: biomass pretreatment and SSF.

In certain exemplary embodiment the pretreated solids by autohydrolysis at S₀=1,82, Tₘₐₓ=130° C. are employed in SSF assays. The SSF experiments were carried out in an orbital incubator using an LSR of 8 g/g of pretreated U. lactuca, at 35° C., pH=5 and 150 rpm. The enzymes used are CellicCTec2 and Viscozyme. The cellulase activity of CellicCtec2 was measured by Filter Paper Assay (explained in section 2.5. with enzyme activity also described in the later section). SSF assays were carried out at enzyme to substrate ratio of 20 FPU/g for CellicCTec2. The ratio of Viscozyme to CellicCTec2 is 5 U/FPU. For this step, no additional nutrients, nor commercial supplementation (peptone and yeast extract) are added because the composition analysis of U. lactuca revealed high protein content, which provides a great quantity of nitrogen in order to diminish the cost of the process.

Samples are taken during the SSF progress at desired times, centrifuged at 5000 rpm for 10 min, filtered through 0.2 mm membranes and analyzed via HPLC for sugars (glucose, galactose+mannose) and ethanol concentration.

The results of the SSF are expressed in terms of ethanol yield (%) using the value of ethanol obtained by SSF compared to a potential optimum value of ethanol calculated from the glucan content of the raw U. lactuca among with another factor.

In certain embodiments, each enzyme possesses a different profile or range of molecule hydrolysis as each break down different linkage of polysaccharides. Therefore, for the following enzymatic treatment, the choice of enzyme blend quantity was set according to the composition of U. lactuca. Enzymes cocktails that have shown to be highly efficient such as CellicCtec2 is employed. This treatment converts as much of the glucan to glucose as possible.

In certain exemplary embodiment, enzymatic hydrolysis is performed at 50° C. in an orbital agitator at 150 rpm and pH 5 that is set using a 0.05 N citric acid-sodium citrate buffer. Enzyme cocktail CellicCtec2 and Viscozyme provided by Novozymes, (Denmark) are employed. Cellulase activity is reported following the Filter Paper Assay and expressed in terms of Filter Paper Units (FPU). Viscozyme's polygalacturonate activity is measured relative to the amount of D-galacturonic acid formation from 0.5% w/v polygalacturonic acid in 50 mM sodium acetate buffer (pH 5) following the DNS method. The amount of enzyme that catalyzes the formation of D-galacturonic acid per minute at pH 5 and 37° C. defines the unit of enzymatic activity (U). The enzyme activities for CellicCtec2 and Viscozyme are 160 FPU/mL, and 4206 U/mL respectively. The mixture of enzymes has a synergistic effect, which allows a higher yield of monosaccharides content than using only one enzyme at the time.

In one further exemplary embodiment the enzymatic hydrolysis is carried out in the most favorable conditions possible. These optimal conditions are LSR of 20:1 (or consistency of C=4.76 kg of solid/100 kg of total weight, o.d.b.); enzymes to substrate ratio, ESR=20 FPU/g; Viscozyme to CellicCtec2 ratio, VCR=5U/FPU, temperature, T=50° C.; pH, 5; agitation, 150 rpm.

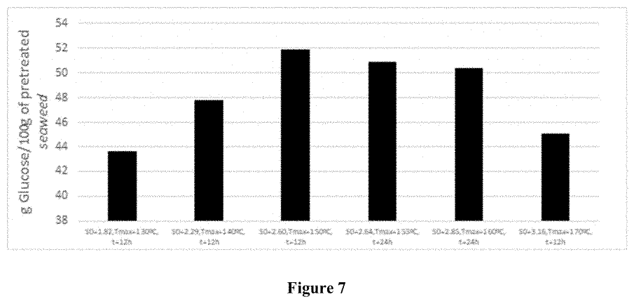

From FIG. 7 the maximum value of glucose recorded at time t during enzymatic hydrolysis was measured in terms of percentage from the initial pretreated biomass of U. lactuca. The value of glucose shown here represent the percentage of glucose that was recovered from the pretreated biomass.

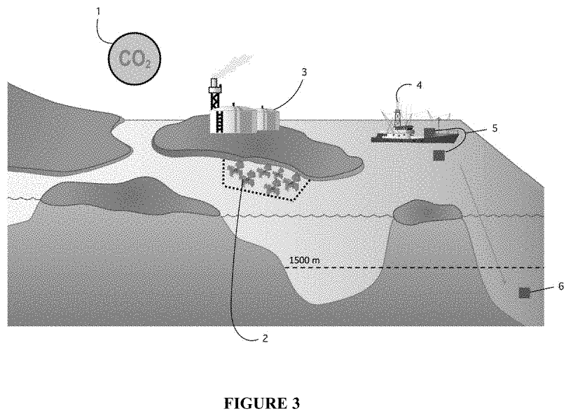

Generally, all pretreated biomass was transformed into glucose with values ranging from 43.59 g of to 51.86 g of glucose per 100 g of pretreated biomass fallowing a parabolic pattern. The pretreatment S0=2.60, (Tmax=150° C.) released the most glucose with 51.86% of the initial pretreated biomass transformed into glucose (see FIG. 3.5). The lowest transformations into glucose occurred for S0=1.82, (Tmax=130° C.), with a value of 43.59% and S0=3.16 (Tmax=170° C.) with a value of 45.0%.

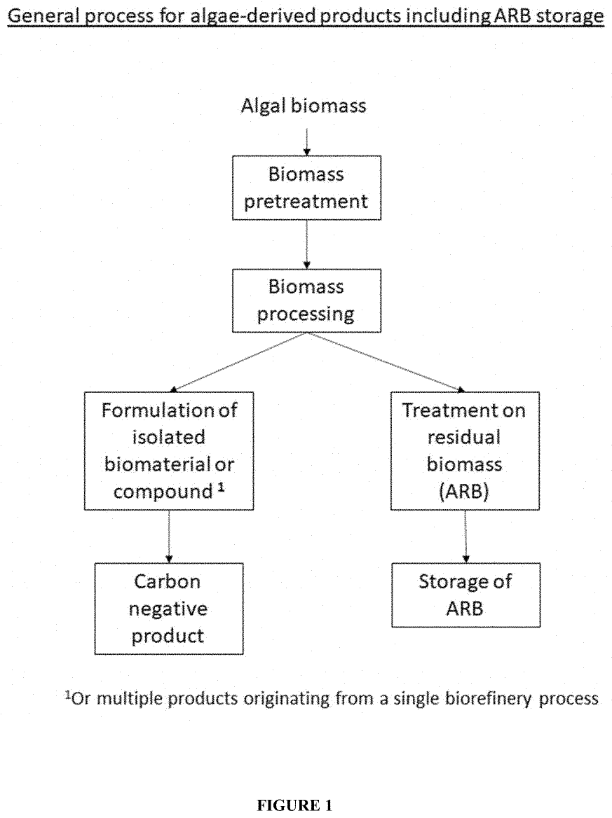

It can be noted from FIG. 1 that all maximum values of glucose released during enzymatic hydrolysis occurred at t=12 h except for S0=2.64 (Tmax=155° C.) and S0=2.85 (Tmax=160° C.).

In certain embodiments Ulva lactuca, a globally distributed macroalgae, that contains a high amount of carbohydrates is used as a raw material for the production of liquid biofuel using a green hydrothermal pretreatment that uses an autohydrolysis process followed by enzymatic hydrolysis. Pretreated biomass at severity S0=2.64 (Tmax=155° C.), followed by 12 h enzymatic hydrolysis allows a maximum of 57% transformation by hydrolysis of its total weight into glucose corresponding to a 112% glucose conversion efficiency. At pretreatment severity S0=1.82, (Tmax=130° C.) and simultaneous saccharification and fermentation (SSF), a maximum of 17% of the pretreated biomass weight transformation into ethanol (82% conversion efficiency with 0.166 g(EtOH)/g pretreated U. lactuca). These results are much higher than those previously reported. Yet although pretreated solid biomass allowed high ethanol yield, its low solid recovery (±30%) obtained in this study after pretreatment require the use of filtration methods for the recovery of ARB from U. lactuca. Total phenolic compounds (TPC) and Trolox eq (TE) recovery in the liquid phase after pretreatment at S0=3.16 (Tmax=170° C.) showed comparable result as other extraction methods of U. lactuca. This reveals a potentially promising strategy to reduce the overall processing costs of bioethanol.

In certain embodiments It can be noted that the ethanol yield obtained in this study from the pretreated U. lactuca is much higher than the prior art, with a value of 0.166 g(EtOH)/g of pretreated U. lactuca and once more reveals the high potential of use of a pretreatment for the production of biofuel form macroalgae once a pretreatment allowing more pretreated solid recovery is established.

In certain embodiment, the bioethanol and the use of algae biomass for the production as described in the herein invention of bioethanol possesses many advantages, which aid in solving the previously stated issues due to the fact that its biomass grows in a marine environment instead of a terrestrial one. Although the ethanol yield of macroalgae is usually lower than that of terrestrial plants (with ethanol yield circa 90 liters per ton of seaweed, corresponding to 0.07 g(EtOH)/g seaweed), their reproduction yield is much faster. The potential ethanol yield of seaweed therefore accounts for a much higher value than terrestrial plants, having a potential production value of 23 400 L of bioethanol per hectare per year (3.5 fold the bioethanol production yield of sugarcane and 11 fold the bioethanol production yield of maize.

The liquid was distilled using a rectification column and three successive pervaporation steps have been performed, the first two using an organophilic PDMS membrane, collecting concentrated ethanol in the permeate and the third with a hydrophilic PVA membrane for water extraction.

Membranes used were either PERVAPTM 4060 (PDMS) or PERVAPTM 4100 (PVA) purchased from DeltaMem AG. Pervaporation modules used were PervaFlow modules from Secoya Tech, with surface exchanges of 0.0169 m2 (PervaFlowV1, 6 mL inner volume) or 0.0024 m2 (PervaFlowV2 mini, 360 μLmL inner volume).

For all experiments, liquid feeding was ensured using a volumetric pump Knauer P4.1S. Vacuum was maintained using a Büichi Vac V500 diaphragm pump. Pressure in the vacuum channel was monitored with a Büichi vacuum controller V800. PervaFlowV1 heating is maintained using a heating plate Heidolph MR 3003, PervaFlowV2 mini heating is performed through immersion within a heated bath.

For each step, the pervaporation module is put under vacuum and heated upon target temperature. Once at temperature, continuous feeding is undergone until exhaustion on the feeding solution. Vacuum pressure and module internal temperature are noted over processing. Pervaporation module emptying is performed with air at a flowrate matching the feeding flowrate. Permeate condensation and collection is performed between the module and the vacuum pump, and after the vacuum pump. Collected permeate phases are gathered afterward. Retentate is first brought down to room temperature before collection.

Density measurements are performed by weighing 1 mL of sample taken using a Hamilton syringe. The first two steps were performed sequentially with a PERVAPTM 4060 (PDMS) hydrophobic membrane installed within a PervaFlowV1 module. The permeate, enriched in ethanol, was collected, and used as feed for the subsequent step. For the third step, a PERVAPTM 4100 (PVA) hydrophilic membrane was used with a PervaFlowV2 mini module. Here, the retentate is enriched in ethanol.

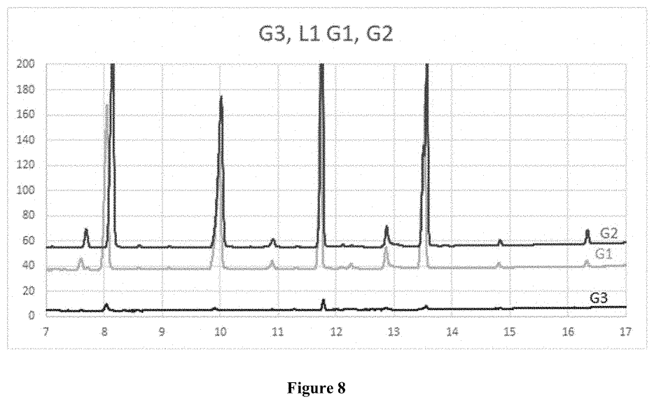

In such embodiment the ethanol obtained after phase 3 (G3) is anhydrous ethanol is obtained as showed in FIG. 8.

In certain embodiments, the extraction and recovery of algae bioactive compounds are carried out by techniques including supercritical fluid extraction, ultrasound assisted extraction, microwave assisted extraction and enzymatic assisted extraction. In other embodiments, bioactive compounds are also extracted by maceration or percolation. Finally, bioactive compounds are extracted and recovered by Soxhlet, liquid—solid extraction, liquid—liquid extraction and concentrated to be recovered due to their toxic properties.

In certain embodiments, the extraction and recovery of algae bioactive compounds are carried out by techniques including supercritical fluid extraction, ultrasound assisted extraction, microwave assisted extraction and enzymatic assisted extraction. In other embodiments, bioactive compounds are also extracted by maceration or percolation. Finally, bioactive compounds are extracted and recovered by Soxhlet, liquid—solid extraction, liquid—liquid extraction and concentrated to be recovered due to their toxic properties.

For example, in preferred embodiment, the solvent used is ethanol.

Components may be selectively isolated by varying the ratio of solvent. Proteins can be extracted from an algal biomass with about 50% ethanol, polar lipids with about 80% ethanol, and neutral lipids with about 95% or greater ethanol. If methanol were to be used, the solvent concentration to extract proteins from an algal biomass would be about 70%. Polar lipids would require about 90% methanol, and neutral lipids would require about 100% methanol.

Exemplary embodiments may be applied to any algae or non-algae material. Exemplary embodiments may use any water-miscible slightly nonpolar solvent, including, but not limited to, methanol, ethanol, isopropanol, acetone, ethyl acetate, and acetonitrile. Specific embodiments preferably use a green renewable solvent, such as ethanol. The alcohol solvents tested resulted in higher yield and purity of isolated neutral lipids. Ethanol is relatively economical to purchase as compared to other solvents disclosed herein. In some exemplary embodiments, extraction and fractionation can be performed in one step followed by membrane-based purification if needed. The resulting biomass is almost devoid of water and can be completely dried with lesser energy than an aqueous algae slurry.

In some exemplary embodiments, the solvent used to extract is ethanol. Other embodiments include, but are not limited to, cyclohexane, petroleum ether, pentane, hexane, heptane, diethyl ether, toluene, ethyl acetate, chloroform, dicholoromethane, acetone, acetonitrile, isopropanol, and methanol. In some embodiments, the same solvent is used in sequential extraction steps. In other embodiments, different solvents are used in each extraction step. In still other embodiments, two or more solvents are mixed and used in one or more extraction steps.

In some embodiments of the methods described herein, a mixture of two or more solvents used in any of the extraction steps includes at least one hydrophilic solvent and at least one hydrophobic solvent. When using such a mixture, the hydrophilic solvent extracts the material from the biomass via diffusion. Meanwhile, a relatively small amount of hydrophobic solvent is used in combination and is involved in a liquid-liquid separation such that the material of interest is concentrated in the small amount of hydrophobic solvent. The two different solvents then form a two-layer system, which can be separated using techniques known in the art. In such an implementation, the hydrophobic solvent can be any one or more of an alkane, an ester, a ketone, an aromatic, a haloalkane, an ether, or a commercial mixture (e.g., diesel, jet fuel, gasoline).

In some embodiments, the extraction processes described herein incorporate pH adjustment in one or more steps. Such pH adjustment is useful for isolating proteinaceous material. In some embodiments, the pH of the extraction process is acidic (e.g., less than about 5). In some embodiments, the pH of the extraction process is alkaline (e.g., greater than about 10).

In certain embodiments antioxidant compound recovery from the unused liquid phase obtained after pretreatment. Total phenolic compounds (TPC) and Trolox eq (TE) recovery in the liquid phase after pretreatment at S0=3.16 (Tmax=170° C.) showed comparable result as other extraction methods of U. lactuca. This reveals a potentially promising strategy to of co-production of algae compounds as described in the herein invention and more precisely in section [026].

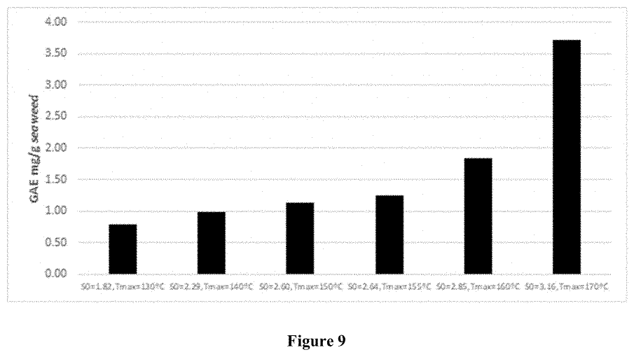

From FIG. 9, it can be noted that GAE values increase exponentially as the severity of the pretreatment increases. The minimum GAE is obtained at S0=1.82, (Tmax=130° C.) with 0.79 mg GAE/g seaweed DW and the maximum is reached at S0=3.16, (Tmax=170° C.) with 3.72 mg GAE/g seaweed DW.

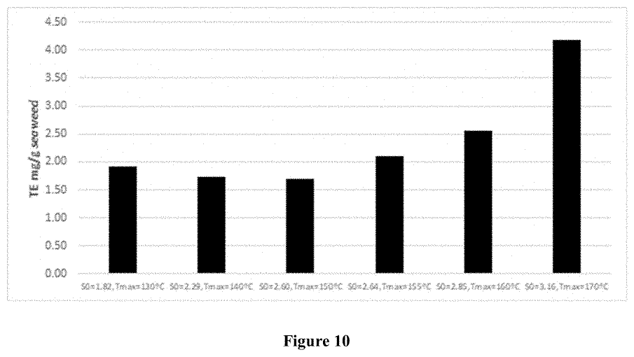

From FIG. 10, it can be noted that the TE value ranges follow a negative parabolic curve as the severity of the pretreatment increases. The TE value ranges from 1.68 to 4.18 mg TE/g seaweed DW. TE values start at 1.92 mg TE/g U. lactuca at S0=1.82, (Tmax=130° C.) and reach a minimum at S0=2.60, (Tmax=150° C.) with 1.68 mg TE/g U. lactuca. Finally, from S0=2.60, (Tmax=150° C.) to S0=3.16 (Tmax=170° C.) TE value increases until it reaches a maximum of 4.18 mg TE/g U. lactuca at S0=3.16 (Tmax=170° C.).

In certain embodiments, proteins are extracted by one or a combination of the following treatments including, but not limited to, aqueous extraction, enzymatic assisted extraction, osmotic shock, spray drying, air classification, hot water buffer, subcritical water extraction, pulse electric field, accelerated extraction, alkali and precipitation amongst other methods. Moreover, proteins can be extracted and recovered by organic solvent or acid. Solvents and acid are recovered by concentration or dehydration.

A further embodiment of the methods and systems described herein is the ability to extract proteins from an algal biomass. For example, in some embodiments, extraction and fractionation occur in a single step, thereby providing a highly efficient process. In certain embodiments, proteins sourced from such biomass are useful for animal feeds, food ingredients, and industrial products. For example, such proteins are useful in applications including, but not limited to, fibers, adhesives, coatings, ceramics, inks, cosmetics, textiles, chewing gum, and biodegradable plastics.

In certain embodiments, lipids are extracted by water extraction, enzymatic assisted extraction, ultrasound assisted extraction, liquefaction, solvents assisted extraction (followed by solvent recovery).

Polar lipid recovery depends mainly on its ionic charge, water solubility, and location (intracellular, extracellular or membrane bound). Examples of polar lipids include, but are not limited to, phospholipids and glycolipids. Strategies that can be used to separate and purify polar lipids can roughly be divided into batch or continuous modes. Examples of batch modes include precipitation (e.g., pH, organic solvent), solvent extraction and crystallization. Examples of continuous modes include centrifuging, adsorption, foam separation and precipitation, and membrane technologies (e.g., tangential flow filtration, diafiltration and precipitation, and/or ultra-filtration).

In certain embodiments, a neutral lipid fraction obtained by the use of the present invention possesses a low metal content, thereby enhancing stability of the lipid fraction, and reducing subsequent processing steps. Metals tend to make neutral lipids unstable due to their ability to catalyze oxidation. Furthermore, metals inhibit hydrotreating catalysts, necessitating their removal before a neutral lipid mixture can be refined. The systems and methods disclosed herein allow for the extraction of metals in the protein and/or the polar lipid fractions. This is advantageous because proteins and polar lipids are not highly affected by metal exposure, and in some cases are actually stabilized by metals.

In certain embodiments of the invention, polar lipids are surfactants by nature due to their molecular structure and have a huge existing market. Many of the existing technologies for producing polar lipids are raw material or cost prohibitive. Alternative feedstocks for glycolipids and phospholipids are mainly algae oil, oat oil, wheat germ oil and vegetable oil. Algae oil typically contains about 30-85% (w/w) polar lipids depending on the species, physiological status of the cell, culture conditions, time of harvest, and the solvent utilized for extraction. Further, the glycerol backbone of each polar lipid has two fatty acid groups attached instead of three in the neutral lipid triacylglycerol. Transesterification of polar lipids may yield only two-thirds of the end product, i.e., esterified fatty acids, as compared to that of neutral lipids, on a per mass basis. Hence, removal and recovery of the polar lipids would not only be highly beneficial in producing high quality biofuels or triglycerides from algae, but also generate value-added co-products glycolipids and phospholipids, which in turn can offset the cost associated with algae-based biofuel production. The ability to easily recover and fractionate the various oil and co-products produced by algae is advantageous to the economic success of the algae oil process.

Embodiments of the systems and methods described herein exhibit surprising and unexpected results. First, the recovery/extraction process can be done on a wet biomass. This is a major economic advantage as exemplary embodiments avoid the use of large amounts of energy required to dry and disrupt the cells. Extraction of neutral lipids from a wet algal biomass is far more effective using the systems and methods of the present invention. The yields obtained from the disclosed processes are significantly higher and purer than those obtained by conventional extractions. This is because conventional extraction frequently results in emulsions, rendering component separations extremely difficult.

In certain embodiments, minerals are extracted with supercritical extraction or other processes. Extraction of the target compound is composed of a plurality of steps. In certain embodiments, the order and the sequence of each steps described can differ. Algae derived product processes mentioned above as well as the list of products in Table 4 should not be interpreted as limiting scope of processes, compounds, and product of the invention but merely to serve as representation of the claims and as basis for teaching one's skill to use the herein invention.

Embodiments of the present invention are superior to those known in the art as they require the use of far less energy and render products suitable for use as fuels as well as foodstuffs and nutrient supplements.

It is contemplated that any embodiment discussed in this specification can be implemented with respect to any method or system of the invention. Furthermore, systems of the invention can be used to achieve methods of the invention.

In certain embodiments, the ARB recovery, transformation and sinkage process described further below is interchangeable with any compound manufacturing as described previously.

In certain embodiments, the phase obtained after the processing and separation of the target compounds with the algal biomass is referred to as algal residual biomass (ARB). The ARB obtained after the processing and separation of the target compounds with the algal biomass is liquid, solid or a combination of both. ARB does not possess chemicals hazardous to the environment as green processes were used and or if any contaminant were produced, they have been extracted similarly to acid dehydration step describe previously. The ARB is recovered at one of the following processes of the following manufacturing strategies A: (I), (II); B: (I), (II); C: (I), (II), (III), (IV) and D: (I), (II), (III), (IV), during multiple processes, or all of the above. In certain embodiments, the chemical composition of the ARB ranges from 70%-100% of algal origin. The preferred level of algal origin in the composition is 95% or above. In certain embodiments, this is achieved by carrying out the ARB recovery at the earliest stage of availability prior to further processing. In certain embodiments, the non-algal chemicals originate from inputs at processing step or from the chemical reactions between algae compounds and added chemical, prior to the ARB recovery stage. In certain embodiments, the quantity of ARB recovered, and its carbon content is recorded.

In certain embodiments, the preferred stage for ARB recovery is either (I) or (II) of any product manufacturing model when the targeted algae compounds and the ARB are separated, resulting in high or complete algae ARB composition. The final form of ARB for the following process is solidly packed with a moisture content ranging between about 1 to about 95% (w/w). The algal compounds present in the liquid ARB are recovered in solid form with moisture content ranging between about 1 and about 95% (w/w). This is collected and transformed into the solid ARB for the next process. The ARB liquid and solid are recovered directly after the target compounds or algae derived compounds are recovered in step II of A, B, C, D preferably, and step C (IV), D (IV).

In certain embodiments, the recovery of the target compound or algae derived compound is carried out by, but not limited to, distillation, filtration, membrane filtration, drying, dialysis, or centrifugation.

In certain embodiments, the ARB solid is recovered by, but not limited to precipitation, filtration, microfiltration, ultrafiltration, nanofiltration, reverse osmosis, centrifugation, screw pressing, hydraulic pressing, sedimentation, flocculation, coagulation, hydro cyclone separation, auger pressing, drying, and air classification.

In certain embodiments, the ARB solid is recovered for the liquid ARB preferably by filtration, membrane separation, microfiltration, ultrafiltration, nanofiltration, reverse osmosis coagulation, evaporation, sun drying, airdrying. The ARB liquid is recovered by but not limited to precipitation, coagulation, flocculation, sedimentation, dissolved air flotation, granular filtration, drying or evaporation.

In other embodiments, the invention encompasses using a freefall sinking system, which is a proprietary device to deploy biomass to the bottom of the ocean. It generally consists of an outer shell of one or several materials including, but not limited to, for example, biodegradable fabrics, non-toxic metal, concrete, or combinations thereof filled with biomass (only biomass or a mix between biomass and minerals). It various embodiments, it comes in a variety of sizes, from example about 0.05 m³ to about 1 m³.

In certain embodiments, the freefall sinking system can be gravity-driven or have an added propulsion method to increase its velocity.

In certain embodiments, the freefall sinking system can have stabilizing fins to insure a vertical drop so that it buries in the sea-floor sediment, or no stabilizing fins so that the device remains on the sea floor not in it.

In certain embodiments, the freefall sinking system can be deployed manually or automatically, via a transport and delivery system (TDS).

In certain embodiments, the TDS consists of a modified cargo container (of any size) with a protruding platform that allows for the freefall sinking system to the dropped overboard with or without human intervention. In certain embodiments, the freefall sinking system is placed into drop position either by a hanging rail or a treadmill system.

This system can be used in any vessel that accepts containers, either commercial ships or dedicated vessels.

In certain embodiments, the recovered ARB is then treated accordingly as described herein.

In certain embodiments, the ARB recovered containing carbon, which is obtained using processes described herein. ARB recovery is used directly wet (I) or in another embodiment under dried from (II) after processes described below. In certain embodiments, dried form corresponds to moisture from about 1 to about 20% (w/w). In certain embodiments, wet form moisture content ranges from about 20 to about 95% (w/w).

In certain embodiments, the ARB is further transformed to a soft mass or rigid pack with volume ranging from 500 mm3 to 15 m3. The preferred volume is 1000 cm3 to 1 m3.

In certain embodiments, the ARB solid is kept wet (embodiment I), it is packed by packaging or using a binding agent.

In certain embodiments, packing of the ARB is carried out with compostable film meeting EU standards EN 13432 or other countries equivalent standards on compostable film, preferably made with algae fiber to increase the algae composition of the ARB pack. In certain embodiments, packing can use hemp bags or organic material such as wooden crates. In certain embodiments, the packing processes include shrink wrapping, vacuum packing, or other commonly used methods to date. In certain embodiments, the quantity of ARB packed is dependent to the packing resistance. In certain embodiments, the thickness of the packing material is adjusted to hold the respective ARB mass of a pack unit.

In certain embodiments, the wet ARB paste is mixed with natural algae-based binding agents such as agar gel or other gel made from proteins, collagen or starch. In certain embodiments, other options are natural polymers including polysaccharides (e.g., cellulose, starch, and gums), polypeptides (proteins like casein, albumin, keratin, and DNA). In certain embodiments, another embodiment uses non-aquatic hazardous binders, such as non-solvent-based adhesives, resins, accrolides resin, sol (except zirconia aluminosilicate refractory ceramic fiber), sealants, natural rubber, rubber, wax, beeswax, mucilages, thickeners such as accrolides, candelilla, guar, gum arabic, karaya, shellac, tragacanth, xanthan. In certain embodiments, non-algae based chemical agents are not preferred as they will signify the introduction of a non-native compound into the ocean. The binding agent also contains carbon that adds to the final carbon content to the ARB pack. The packing process with binders is carried out through a molding process or other common method described in previous art.

In certain embodiments, drying (II) can be performed with either one or a combination of the following machines, but not limited to, evaporation, spray dryer, freeze-dryer, sun-dryer, airdried, tray dryer, rotary dryer, drum dryer, cone screw dryer, double cone dryer, sphere dryer, sludge dryer, granulation dryer and fluid bed dryer. Drying brings the moisture content of the ARB down to a range of 1-30% (w/w). The temperature and pressure of the drying procedure are selected to minimize the energy consumption of the process. In another embodiment, the ARB is mechanically compressed after the drying process to reach a density in the range of 400-1700 kg/m-3 with preferred density ranging from 600-1400 kg/m-3. Compression is performed with one or a combination of the following machines including, but limit to, hydraulic press, forging press, crank press, eccentric press, knuckle joint press, extruder, pelletizer, pellet press, pellet mill, grinder and shredder, briquette press. Preferred methods of drying are sun dried, air dried or tray dried as they are energy efficient and possess a low carbon footprint.

In certain embodiments, strategy (I) or (II) is used, the final ARB pack density in the water column is significantly superior to the ocean seawater density (1024 Kg/m3) and sink to the ocean floor. The pre-dissolution time is the time before the packed ARB start to dissolve in seawater. Low density ARB packs have a longer sinking rate and must possess a longer pre dissolution time. It is preferred to use higher density ARB packing that enable a higher sinking rate and afford less dissolving property agents and or packaging material. In certain embodiments, the pre-dissolution time is inferior or equal to the time taken for the ARB pack to sink to 1500 m depth.

The packed ARB's carbon content is analyzed by laboratory analysis, using HPLC, elemental analyzer or other methods, to record the amount of carbon stored and evaluate the net carbon footprint reduction of a product manufacturing process, as well as the feasibility of the herein invention, especially for the manufacturing process that results in little recovery of ARB as previously described.

ARB packs are transported by roadways, rail ways, sea ways, airways, or a combination of a plurality (seaways being the preferred transport especially when using empty fishing vessels going to sea) and placed, dropped, dispersed, propelled, within an ocean, sea, or water zone where no volcanic activity is recorded (such as hydrothermal vents) and the depth ranges from 1000-1500 m. In another embodiment, a shallower depth is used if carbon leaking to the surface from ARB is proven nonexistent. Using the ARB wet pack (embodiment I), when compared to ARB dry pack (embodiment II), requires more energy for transport due to higher water content but remains the less energetic method compared to ARB dry packing process (embodiment II) using energetic machinery, or b) remains more feasible, especially for a large-scale system compared to the ARB dry packing process (embodiment II) using an air-dry system. This is the preferred method.

In a certain embodiment, for the carbon to be sequestered in the ocean for a long period of time, the carbon contained in the biomass must reach at least 1500 m depth. In order to sequester carbon in the ocean, algae can be used as they contain circa 25% carbon in their composition. In a natural ecosystem, algae naturally sequester carbon when they die. Dead algae sink to the ocean floor (called marine snow in biology) as part of what is call the biological pump of the ocean, and more precisely through the particulate organic carbon (POC) flux.

The hydrothermal process of the biomass as described in previous embodiment, breaks the algae's cellular wall. This process allows the density of the waste biomass to increase and helps for its sinkage to the ocean floor.

In other embodiment, other biochemical process described previous embodiment allow the same as [00332] just above.

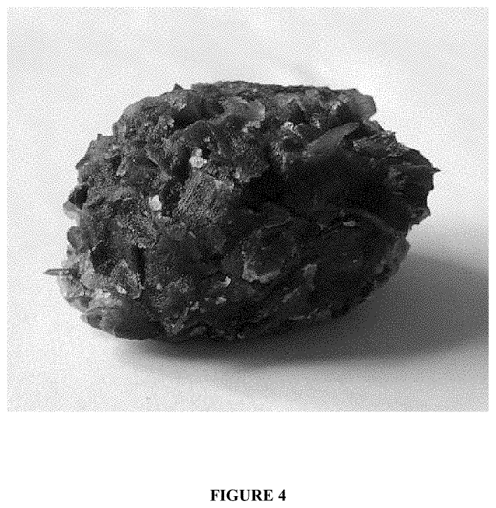

As primary embodiment the ARB is packed and transferred to a sea vessel to be dropped to the deep-sea where the ocean floor is more than 1500 m depth in the bathyal benthic province for example as shown in FIG. 4. This strategy hence allows the long-term capture of CO2 from the atmosphere.

In certain embodiment, the carbon stored is embedded in the end product for the manufacturing of the algae compounds extracted. and offers consumers the chance to buy a product which is carbon negative.

In a certain embodiment, while the concept of seaweed sinkage for carbon sequestration using seaweed already exists and is in use, the herein innovation methods of application this concept using ARB in a circular system of co algae product manufacturing is novel.

The invention provides the first extraction small portion of the algae content and produce consumer products before sequestering the remaining seaweed biomass (ARB).

In certain embodiments, strategy (I) is the preferred and recommended method, especially for large scale operations where strategy (II) might be challenging in terms of operating space. The carbon content of one ARB pack is quantified using elemental analysis HPLC (or other methods) and the carbon content value obtain of one ARB pack is higher than its sinking operation carbon emission. The ARB pack carbon mass ranges from 1 g to 1000 kg. The preferred ARB pack carbon mass ranges from 2-40 g.

In certain embodiments, the distance of drop location of one ARB pack to other ranges from 0-500 m, with preferred seafloor area ranging from 8-16 m2 (relative to the preferred carbon mass mentioned in previous paragraph, resulting in carbon input of 0.25-2.5 g/m2 to the ocean bed, which correspond to predicted depleted carbon quantities reaching the ocean floor by 2100, and therefore does not possess just as CDR technology but a Conservative & CDR technology, or (CCDR). A large quantity of ARB introduced in one location in the ocean can greatly modulate the benthic biogeochemistry and ecology geosystem that is not recommended in this herein method. Smaller quantities of ARB spread over a larger distance is the preferred method.

In other embodiments, the invention encompasses a cyclic carbon dioxide removal (CRD) method. In certain embodiments, the invention encompasses methods for the removal of CO₂ from the atmosphere and disposal of captured carbon dioxide in the algal residual biomass in the deep ocean. In certain embodiments, the invention encompasses various CDR approaches including, but not limited to, bioenergy with carbon capture and storage that increase the burial rate of organic carbon. In certain embodiments, the systems and methods of the invention includes a circular CDR method that accomplishes both conservation and carbon dioxide removal using, for example, range specific ARB spreading to diffusely distribute the ARB in the deep ocean.

A financial instrument tradable under a greenhouse gas Emissions Trading Scheme (ETS) may be created by exploitation of the processes of the present invention. The instrument may be, for example, one of either a carbon credit, carbon offset or renewable energy certificate. Generally, such instruments are tradable on a market that is arranged to discourage greenhouse gas emission through a cap-and-trade approach, in which total emissions are capped, permits are allocated up to the cap, and trading is allowed to let the market find the cheapest way to meet any necessary emission reductions. The Kyoto Protocol and the European Union ETS are both based on this approach. One example of how credits may be generated is as follows. A person in an industrialized country wishes to get credits from a Clean Development Mechanism (CDM) project, under the European ETS. The person contributes to the establishment of plant employing the processes of the present invention. Credits (or Certified Emission Reduction Units, “CERs”) where each unit is equivalent to the reduction of one metric ton of CO₂ or its equivalent) may then be issued to the person. The number of CERs issued is based on the monitored difference between the baseline and the actual emissions. It is expected by the applicant that offsets or credits of a similar nature to CERs will be soon available to persons investing in low carbon emission energy generation in industrialized nations, and these could be similarly generated

A more complete understanding of the present invention will be provided in relation to the following examples which are understood to be non-limiting to the basic inventive concepts of the present invention. The monitoring of the ARB is achieved by a system according to the ISO international standards.

In certain embodiment where the ARB pack in not compressed but simply packed, the ARB sinks at a velocity of 1.96 cm. s⁻¹. At such sinking velocity it takes 21h for the compressed cube to reach 1500 m depth and 2.8 h to reach the mesopelagic zone.

In certain embodiment where the ARB pack is compressed or process an aerodynamic shape, the ARB pack.

In another embodiment empty fishing vessel are used to disperse the biomass.

In certain embodiment the sea vessels are equipped with counter rotation wheels where an electric fan draws in outside air which is then pushed into the canister. To protect the fan motor from debris the air passes through a slab of foam as well as a screen barrier. The counter rotating wheels are used to spread ARB packs in corresponding area of ocean surface area as relative to ocean bed surface to fit the range of ARB input described herein. Other similar machinery described I prior arts can be used for such embodiment.

In certain embodiment, the production of 48 hectoliters of anhydrous ethanol per year production (large scale production), it would result in the sinking of 216 tons of carbon per year.

A more complete understanding of the present invention will be provided in relation to the following examples which are understood to be non-limiting to the basic inventive concepts of the present invention. The monitoring of the ARB is achieved by a system according to the ISO international standards.

### EXAMPLES

### Example I

Ethanol Manufacturing

Wet macroalgae blend (50 kg) was used as a raw material to produce ethanol via fermentation. The algae blend was composed of 70% green algae, Ulva lactuca, and 30% brown algae, Sargassum muticum. U. lactuca was obtained fresh from a seaweed farm in north Portugal, under controlled conditions and S. muticum was freshly collected from the coast of Sagres, Portugal. Despite the specific mix, for the purposes of this process, any algal material can be used. The chemical composition of the macroalgae blend, which possessed 7.1% dry weight residue at 105° C. is provided in Table 8.

TABLE 8
Main chemical compounds of the algae blend was
composed of 70% green algae, Ulva lactuca,
and 30% brown algae, Sargassum muticum.

| | g/100 g oven macroalgae blend ± |
|---|---|
| Components | standard deviation |
| Proteins | 15.76 ± 0.25 |
| Ashes | 19.4 ± 0.07 |
| Lipids | 0.6 |
| Total polysaccharides | 64.2 |

Wet macroalgae biomass were ground into particles using a homogenizer. A hydrothermal pretreatment was used to disrupt the cellular wall of the feed's cells for the recovery of glucan. This was achieved by heating the feed water mixture to a high temperature. The high temperature increased the catalytic action of hydronium ions and organic acid present to degrade the feed. Water and feed were mixed at a consistency 10% w/v in a stainless-steel Pressure bioreactor fermenter. Enzymatic blend was used to hydrolyze the polysaccharide to fermentable sugars. The fermentation process was carried out using Saccharomyces cerevisiae yeast. Once the fermentation is complete, the solid and liquid phase were separated using a screw press. The solid phase (ARB) contains unfermented sugars, uronic acids, proteins, and others insoluble residue. The liquid phase contains ethanol, higher ethers, lipids, minerals, and uronic acid. The liquid phase is used for the ethanol recovery by (distillation, pervaporation, hybrid system) to produce hydrous or anhydrous ethanol (>99.7

The remaining liquid phase was filtered by ultrafiltration to recover solids as ARB. In certain embodiments, the solid ARB is a paste, which was air dried.

The bags were transported off the coast of Portugal in a depth where the ocean is 1,500 m and dropped 20 m apart. The energetic demand of the overall process was monitored. The overall CF balance of the manufacturing of ethanol and carbon permanently stored was calculated and presented in Table 9.

TABLE 9
Summary of main components stream obtained.

| Components | Value | Unit |
|---|---|---|
| Initial wet biomass | 50 | kg |
| Dry weight algae | 7.5 | kg |
| Carbon content algae | 2.4 | kg |
| Solid loading | 10 | % w/v |
| Ethanol yield | 0.1 | g EtOH/g |
| | | DW* algae |
| Carbon content ARB | 23 | % |
| Mass of the wet ARB | 40 | kg |
| Mass of the packed ARB | 1 | kg |
| Mass of Carbon input into ocean floor per ARB | 46 | g C |
| pack | | |
| Sinking duration to reach 1500 m depth | 21 | h |
| Net CO₂ Emission for the manufacturing and ARB | 6.88 | Kg CO₂/L |
| storage operation per volume of hydrous ethanol | | |
| Net CO₂ eq permanently stored per volume | −8.46 | Kg CO₂/L |
| of hydrous ethanol | | |
| Net Overall CO₂ balance | −1.57 | Kg CO₂/L |

DW* = Dried Weight

### Example II

Protein Manufacturing

Using the same conditions and biomass as example 1, electroporation of the macroalgal blend was performed with a batch electroporator. The pulsed electrified field (PEF) chamber was loaded with the blend in order to reach a biomass concentration of 10 g_{DW} L⁻¹ in a 0.05% sodium chloride solution having a conductivity of 1240 μS cm⁻¹. The electric field applied in the chamber had a strength equivalent to 7 kV cm⁻¹ with 0.1 ms pulses. Subsequently to the PEF treatment, the biomass was left in the chamber for t=120 min in order to maximize the release of proteins and carbohydrates in the solution. The solution and the ARB separate spontaneously by sedimentation therefore the solution is treated with dialysis against deionized water (MWCO 500 Da) followed by freeze-drying in order to obtain a protein powder. The protein yield obtained with this method is 14.6%. A dry weight loss equal to 11% in the residual biomass were measured, and show that other components (mainly, ashes and carbohydrates) have been extracted and solubilized during the PEF treatment.

The wet ARB carbon content was measured. The ARB paste was sealed in 1 kg compostable film pack. The sealed ARB bags density and sinking rate were measured empirically and results presented in Table 10. The degradation of the compostable bag was measured empirically at the ocean surface using a transparent incubating chamber.

The bags were transported off the coast of Portugal to a depth where the ocean is 1500 m and dropped 20 m apart. The energetic demand of the overall process was monitored. The overall CF balance of the manufacturing of ethanol and carbon permanently stored was calculated and presented in Table 10.

TABLE 10
Summary of main components stream obtained.

| Components | Value | Unit |
|---|---|---|
| Initial wet biomass | 50 | kg |
| Dry weight algae | 7.5 | kg |
| Carbon content algae | 2.4 | kg |
| Solid loading in PEF chamber | 10 | g DW · L⁻¹ |
| Protein yield | 14.6 | % |
| Carbon content ARB (DW) | 27 | % |
| Mass of the wet ARB | 48.15 | kg |
| Mass of the packed ARB | 1 | kg |
| Mass of Carbon input into ocean floor | 46 | g C/m² |
| (1 bag of ARB) | | |
| Sinking duration to reach 1500 m depth | 21 | h |
| Net CO₂ Emission for the manufacturing and | 10.77 | Kg CO₂/kg |
| ARB storage operation per kg of proteins | | |
| Net CO₂ eq permanently stored per kg of proteins | 30.1 | Kg CO₂/kg |
| Net Overall CO₂ balance | −19.33 | Kg CO₂/kg |

While the present invention has been specifically described with respect to separation and recovery of carbon dioxide, it will be appreciated that the present invention may be readily used to separate other gases.

It is to be understood that, although prior art use and publications may be referred to herein, such reference does not constitute an admission that any of these form a part of the common general knowledge in the art, in Australia or any other country.

Numerous variations and modifications will suggest themselves to persons skilled in the relevant art, in addition to those already described, without departing from the basic inventive concepts. All such variations and modifications are to be considered within the scope of the present invention, the nature of which is to be determined from the foregoing description.

1. A method for manufacturing algae-compound-derived products and algae-derived compound products that reduce, neutralize, or negate the overall carbon footprint of those products, comprising:
a. farming a plurality of algae or wild harvesting a plurality of algae;
b. transformation of the plurality of algae compounds by a plurality of chemical physical process into a plurality of compound products;
c. recovery of unused algae residual compounds obtained as a byproduct of the processing of the plurality of harvested algae step b;
d. packing of the (ARB) by binding gel, packeting fiber, or drying (with optional compression process);
e. transport of the ARB packs by sea ways, roadways, rails ways, air ways and its sinking to an ocean zone where the depth is below 1000 m;
f. tracing and recording in a database all the greenhouse gas emissions from steps a, b, c, d, e.

2. The method of claim 1, wherein the products apply to any industry sectors such as bioethanol for various industries which comprises fuel, consumer products, spirits, perfume, cosmetics which includes face cream, hair gel, any types of cream for skin, hair, shampoos.

3. The method of claim 1, wherein the product carbon footprint status (neutral, negative and reduced) is established by the current and future international ISO standards or the corresponding current of future country's legislation in which this manufacturing method is applied.

4. The method of claim 1, wherein a portion of the product's net negative carbon value can be used as stream revenue from carbon credit scheme as to reduce the green premium cost of the respective product manufacturing.

5. The method of claim 1, that using energy saving methods for steps a, b, c, d, e result in increased negation power of the product carbon footprint according to the standards.

6. The method of claim 1, wherein the method of manufacturing ethanol and ocean carbon dioxide removal (CDR) generate a carbon offset by the removal of atmospheric carbon dioxide and permanent disposal of the residual algal biomass, while simultaneously avoiding production of atmospheric carbon dioxide typically emitted during the manufacture of ethanol.

7. The method of claim 1, wherein step a. algae understands microalgae, macroalgae, and blue green algae: cyanobacteria; freshwater algae species, in a separated or simultaneous cultivation system (multispecies cultivation).

8. The method of claim 1, wherein step a. includes farming the algae in non-arable land or in the ocean to prevent the withdrawal of arable spaces for food crop.

9. The method of claim 1, wherein step a. includes farming the algae with non-intensive techniques consisting of: wild cultivation, ocean aquaculture, open ponds, collaboration with fish farm in circular aquaculture system (other aquaculture species growth: intended to optimize the overall carbon footprint of the product (according to its ISO inventory scope);

10. The method of claim 1, wherein step a. includes farming the algae with intensive techniques intended to optimize productivity and selected from the group consisting of: ocean aquaculture, open ponds, vertical or horizontal tubular photobioreactor, flat panel airlift photobioreactor and bubble column photobioreactor.

11. The method of claim 1, wherein step a. includes farming the algae in sea water or freshwater.

12. The method of claim 1, wherein step a. includes genetically modifying and or use of genetically modified plurality of algae to improve the photosynthetic efficiency.

13. The method of claim 1, wherein step a. includes strain selective and or use of strain selective plurality of algae to improve the photosynthetic efficiency.

14. The method of claim 1, wherein step a. and c. can be both located or separately located on land, coastal or offshore area, on the surface water or above on platforms.

15. The method of claim 1, wherein step b. includes the isolation and purification of a plurality of compounds present in the plurality of harvested algae

16. The method of claim 12, includes the use of the plurality of algae compounds isolated, purified or extracted by chemical physical process for further chemical physical chemical process into a plurality of algae-derived compound products.

17. The method of claim 13, wherein its step further includes the use of the plurality of algae-derived compound products in order to make a plurality of commercial products as part of a formulation scheme.

18. The method of claim 1, wherein step d. includes testing the safety of the formulated plurality of compounds which are intended for human, animals or plants consumption or use.

19. The method of claim 1, wherein step c. includes the recovery of unused algae residual compounds in any steps process mentioned claimed 1 and 12-14 included.

20. The method of claim 1 comprising any of or a combination of precipitation, filtration, microfiltration, ultrafiltration, nanofiltration, reverse osmosis, centrifugation, screw pressing, hydraulic pressing, sedimentation, flocculation, coagulation, hydro cyclone separation, auger pressing, drying, air classification, coagulation, evaporation, sun drying, airdrying, dissolved air flotation or granular filtration;

21-34. (canceled)
